(19) United States
(12) Patent Application Publication
(10) Pub. No.: US 2024/0279395 A1
(43) Pub. Date: Aug. 22, 2024

(54) FLUORINE-CONTAINING ETHER COMPOUND, LUBRICANT FOR MAGNETIC RECORDING MEDIUM, AND MAGNETIC RECORDING MEDIUM

(71) Applicant: Resonac Corporation, Tokyo (JP)

(72) Inventors: Natsumi SHIBATA, Tokyo (JP); Naoya FUKUMOTO, Tokyo (JP); Ayano ASANO, Tokyo (JP); Tsuyoshi KATO, Tokyo (JP); Takuma KURODA, Tokyo (JP); Daisuke YAGYU, Tokyo (JP); Chizuru KASAHARA, Tokyo (JP); Yutaka TANJI, Tokyo (JP)

(73) Assignee: Resonac Corporation, Tokyo (JP)

(21) Appl. No.: 18/285,609

(22) PCT Filed: Apr. 7, 2022
(86) PCT No.: PCT/JP2022/017256

§ 371 Date: Oct. 4, 2023

(30) Foreign Application Priority Data
Apr. 8, 2021 (JP) 2021-065868

(51) Int. Cl. C08G 65/22; C10M 107/38; C10N 40/18

(52) U.S. Cl. CPC C08G 65/226; C10M 107/38; C10N 2040/18

(57) ABSTRACT

The present invention provides a fluorine-containing ether compound represented by following formula.

R¹—CH₂—R²—CH₂—R³—CH₂—R⁴—CH₂—R⁵—CH₂—R⁶—CH₂—R⁷

(R², R⁴, and R⁶ are perfluoropolyether chains having the structure R³ and R⁵ are each independently a linking group containing one or more hydroxyl groups, and R¹ and R⁷ are each independently Formula (2).)

—O—CH₂—CH(OH)—([D]—CH(OH))ₛ-[E]-CH₂OH (2)

(s is 0 or 1, [D] and [E] each independently is a chain structure consisting of a combination of 2 to 5 methylene groups and 1 oxygen atom or a chain structure consisting of 1 to 4 methylene groups, provided that, in case where s is 0 and [E] contains an oxygen atom, the number of methy ne groups contained in [E] is 3 or more).

### TECHNICAL FIELD

The present invention relates to a fluorine-containing ether compound, a lubricant for a magnetic recording medium, and a magnetic recording medium.

Priority is claimed on Japanese Patent Application No. 2021-065868, filed Apr. 8, 2021, the content of which is incorporated herein by reference.

### BACKGROUND ART

Development of magnetic recording media suitable for high recording densities is underway to improve the recording densities of magnetic recording/reproducing devices.

As a conventional magnetic recording medium, there is a magnetic recording medium in which a recording layer is formed on a substrate and a protective layer made of carbon or the like is formed on the recording layer. The protective layer protects information recorded in the recording layer and enhances the slidability of a magnetic head. In addition, the protective layer covers the recording layer to prevent metal contained in the recording layer from being corroded by environmental substances.

However, sufficient durability of the magnetic recording medium cannot be obtained by simply providing the protective layer on the recording layer. Therefore, a lubricant is applied to the surface of the protective layer to form a lubricating layer with a thickness of about 0.5 to 3 nm. The lubricating layer improves the durability and protective power of the protective layer and prevents contamination substances from intruding into the magnetic recording medium.

After forming the lubricating layer on the surface of the protective layer, a burnishing step may be performed to remove projections and particles present on the surface of the magnetic recording medium and improve the smoothness of the surface.

As a lubricant that is used at the time of forming a lubricating layer in a magnetic recording medium, there is, for example, a lubricant containing a fluorine-based polymer having a repeating structure containing —CF₂— and having a polar group such as a hydroxyl group at a terminal.

For example, Patent Document 1 discloses a magnetic disk provided with a lubricating layer containing a fluorine-containing ether compound containing three perfluoropolyether chains in its molecule and having the same structure at both terminals.

Patent Document 2 discloses a magnetic disk provided with a lubricating layer containing a fluorine-containing ether compound containing three perfluoropolyether chains in its molecule and having two different terminal structures.

In addition, Patent Document 3 discloses a magnetic disk having a lubricating layer containing a lubricant containing three perfluoropolyether chains in its molecule and having two hydroxyl groups in linking groups between the perfluoropolyether chains.

### CITATION LIST

### Patent Document

[Patent Document 1]
PCT International Publication No. WO2018/116742
[Patent Document 2]
PCT International Publication No. WO2017/145995
[Patent Document 3]
United States Patent Application, Publication No. 2016/0260452

### SUMMARY OF INVENTION

### Technical Problem

There is a demand for a further decrease in the flying height of a magnetic head in magnetic recording/reproducing devices. This requires a further decrease in the thickness of lubricating layers in magnetic recording media.

However, in general, when the thickness of lubricating layers is reduced, the coatability of the lubricating layers tends to decrease, and the wear resistance of magnetic recording media tend to decrease. For this reason, there is a demand for lubricating layers to have a high wear resistance.

In addition, conventional magnetic recording media may have insufficient corrosion resistance. In particular, in a case where a tape burnishing process is performed on the surface of a magnetic recording medium after forming a lubricating layer, the corrosion resistance of the magnetic recording medium is likely to be insufficient. For this reason, there is a demand for a lubricating layer which is highly effective in suppressing corrosion of magnetic recording media.

The present invention has been made in consideration of the above circumstances, and an object of the invention is to provide a suitable fluorine-containing ether compound as a material for a lubricant for a magnetic recording medium with which a lubricating layer having an excellent wear resistance and effect of suppressing corrosion of a magnetic recording medium can be formed.

In addition, another object of the present invention is to provide a lubricant for a magnetic recording medium which contains the fluorine-containing ether compound of the present invention and with which a lubricating layer having an excellent wear resistance and effect of suppressing corrosion of a magnetic recording medium can be formed.

In addition, still another object of the present invention is to provide a magnetic recording medium in which a lubricating layer containing the fluorine-containing ether compound of the present invention is provided and which has an excellent wear resistance and corrosion resistance.

### Solution to Problem

That is, the present invention relates to the following features.

A first aspect of the present invention provides the following fluorine-containing ether compound.

[1] A fluorine-containing ether compound represented by Formula (1) below.

R¹—CH₂—R²—CH₂—R³—CH₂—R⁴—CH₂—R⁵—CH₂—R⁶—CH₂—R⁷ (1)

(In Formula (1). R², R⁴, and R⁶ are perfluoropolyether chains having the same Structure. R³ and R⁵ are each independently a linking group containing one or more hydroxyl groups, and R¹ and R⁷ are each independently a terminal group represented by Formula (2) below.)

—O—CH₂—CH(OH)—([D]—CH(OH))ₛ-[E]-CH₂OH (2)

(In Formula (2), s is 0 or 1, [D] and [E] each independently is a chain structure consisting of a combination of two to five methylene groups (—CH₂—) and one oxygen atom (—O—) or a chain structure consisting of one to four methylene groups (—CH₂—), provided that, in a case where s is 0 and [E] contains an oxygen atom, the number of methylene groups contained in [E] is 3 or more.)

The fluorine-containing ether compound of the first aspect of the present invention prefer escribed in [2] to [8] below. It is also preferable to arbitrarily con bi scribed in [2] to [8] below.

[2] The fluor d according to [1], in which the terminal group represented by Formula (2) above is a terminal group represented by any of Formulae (2-1) to (2,3) and (3-1) to (3-4) below,

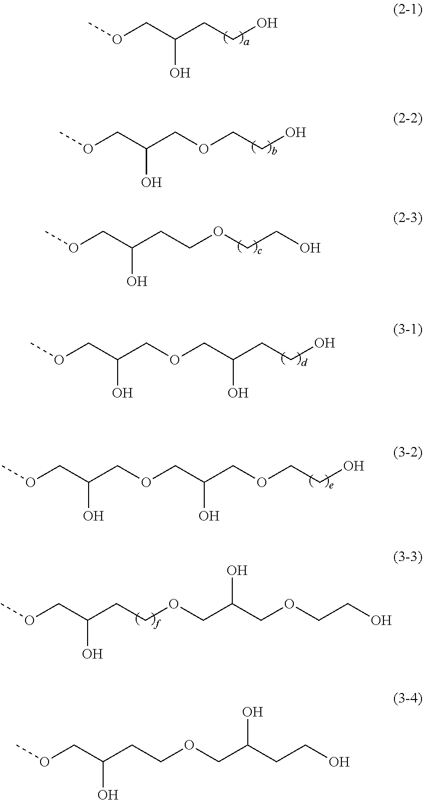

(a in Formula (2-1) is an integer of 1 to 4.)
(b in Formula (2-2) is an integer of 2 to 3.)
(e in Formula (2-3) is an integer of 1 to 3.)
(d in Formula (3-1) is an integer of 1 to 4.)
(e in Formula (3-23 is an integer of 1 to 3.)
(f in Formula (3-3) is an integer of 1 to 2.)

[3] The fluorine-containing ether compound according to [2], in which at least one of R¹ and R⁷ in Formula (1) above is the terminal group represented by any of Formula (3-1) to (3-4) above.

[4] The fluorine-containing ether compound according to any one of [1] to [3], in which R³ and R⁵ in Formula (1) above are linking groups represented by Formula (4) below.

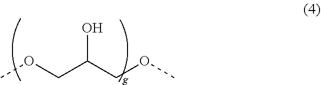

(g in Formula (4) is an integer of 1 to 4.)

[5] The fluorine-containing ether compound according to any one of [1] to [4], in which R², R⁴, and R⁶ in Formula (1) above are any of Formulae (5) to (9) below.

—CF₂O—(CF₂CF₂O)ₕ—(CF₂O)ᵢ—CF₂— (5)

(In Formula (5), h and i indicate the average degree of polymerization and each independently represent 0.1 to 20.)

—CF₂O—(CF₂CF₂O)ⱼ—CF₂— (6)

(In Formula (6), j indicates the average degree of polymerization and represents 0.1 to 20.)

—CF₂CF₂O—(CF₂CF₂CF₂O)ₖ—CF₂CF₂— (7)

(In Formula (7), k indicates the average degree of polymerization and represents 0.1 to 20.)

—CF₂CF₂CF₂O—(CF₂CF₂CF₂CF₂O)ₗ—CF₂CF₂CF₂— (8)

(In Formula (8), l indicates the average degree of polymerization and represents 0.1 to 10.)

—CF(CF₃)O—(CF₂CF(CF₃)O)ᵣ—CF(CF₃)— (9)

(In Formula (9), r indicates the average degree of polymerization and represents 0.1 to 20.)

[6] The fluorine-containing ether compound according to any one of [1] to [5], in which R¹ and R⁷ in Formula (1) above are the same as each other.

[7] The fluorine-containing ether compound according to any one of [1] to [6], in which R³ and R⁵ in Formula (1) above are the same as each other.

[8] The fluorine-containing ether compound according to any one of [1] to [7], in which a number average molecular weight thereof is within a range of 500 to 10,000.

A second aspect of the present invention is to provide a lubricant for a magnetic recording medium below.

[9] A lubricant for a magnetic recording medium including: the fluorine-containing ether compound according to any one of [1] to [8].

A third aspect of the present invention is to provide a magnetic recording medium below.

A magnetic recording medium, in which at least a magnetic layer, a protective layer, and a lubricating layer are sequentially provided on a substrate, and the lubricating layer contains the fluorine-containing ether compound according to any one of [1] to [8].

The magnetic recording medium of the third aspect of the present invention preferably includes features described in below.

[11] The magnetic recording medium according to [10], in which the average film thickness of the lubricating layer is 0.5 nm to 2.0 mm.

### Advantageous Effects of Invention

The fluorine-containing ether compound of the present invention is the compound represented by Formula (1) above, and therefore can be used as a material for a lubricant for a magnetic recording medium with which a lubricating layer having an excellent wear resistance and effect of suppressing corrosion of a magnetic recording medium can be formed.

Since the lubricant for a magnetic recording medium of the present invention contains the fluorine-containing ether compound of the present invention, it is possible to form a lubricating layer having an excellent wear resistance and effect of suppressing corrosion of a magnetic recording medium.

The magnetic recording medium of the present invention is provided with the lubricating layer containing the fluorine-containing ether compound of the present invention, and therefore has an excellent wear resistance and corrosion resistance. For this reason, the magnetic recording medium of the present invention has excellent reliability and durability. In addition, since the magnetic recording medium of the present invention is provided with the lubricating layer having an excellent wear resistance and effect of suppressing corrosion of a magnetic recording medium, the thickness of a protective layer and/or the lubricating layer can be reduced.

### BRIEF DESCRIPTION OF DRAWING

FIG. 1 is a schematic cross-sectional view showing an example of one embodiment of a magnetic recording medium of the present invention.

### DESCRIPTION OF EMBODIMENT

The present inventors have conducted extensive studies focusing on a chain skeleton and terminal groups of a fluorine-containing ether compound to solve the above-described problems.

As a result, they have found that a fluorine-containing ether compound may be used which has a chain skeleton, in which three perfluoropolyether chains having the same structure are bound to each other via a linking structure in which methylene groups (—CH₂—) are bound to both ends of a linking group containing one or more hydroxyl groups, and in which terminal groups each having two or three hydroxyl groups and represented by Formula (2) are bound to both ends of the chain skeleton via methylene groups.

When the lubricating layer containing the fluorine-containing ether compound having the above-described chain skeleton is formed on a protective layer, both ends of the perfluoropolyether chain placed at the center of the chain skeleton adhere closely to the protective layer due to hydroxyl groups in the linking groups arranged between perfluoropolyether chains. For this reason, a lubricant containing the fluorine-containing ether compound having the above-described chain skeleton is likely to wet and spread on the protective layer, can uniformly adhere closely to the protective layer, and can form a lubricating layer with a high coating rate and favorable adhesion properties compared to a case where a lubricant contains a fluorine-containing ether compound having one or two perfluoropolyether chains and having the same number of carbon atoms as the above-described chain skeleton.

In addition, the intramolecular mobility of the perfluoropolyether chains varies depending on structures of the perfluoropolyether chains. Since the fluorine-containing ether compound having the above-described chain skeleton has three perfluoropolyether chains having the same structure, molecular strain due to the difference in the mobility among the perfluoropolyether chains is less likely to occur. For this reason, the fluorine-containing ether compound having the above-described chain skeleton has more favorable molecular linearity than a compound in which perfluoropolyether chains having different structures are mixed in its molecule. As a result, the lubricant containing the fluorine-containing ether compound having the above-described chain skeleton can uniformly adhere closely to the protective layer and can form a lubricating layer with a high coating rate and favorable adhesion properties.

In addition, each terminal group represented by Formula (2) has two or three hydroxyl groups, and the distance between the hydroxyl groups is appropriate.

Moreover, in each terminal group represented by Formula (2), even in a case where a linking group placed between a carbon atom to which the most terminal hydroxyl group (terminal hydroxyl group) is bound and a carbon atom to which a hydroxyl group adjacent to the terminal hydroxyl group is bound contains an oxygen atom, the number of methylene groups contained in the above-described linking group is appropriate.

Therefore, it is inferred that the lubricating layer containing the fluorine-containing ether compound in which the terminal groups represented by Formula (2) are bound to both ends of the above-described chain skeleton would have appropriate hydrophobicity and excellent adhesion properties with respect to the protective layer.

The present inventors have conducted further studies to confirm that, when the lubricating layer containing the above-described fluorine-containing ether compound is formed on the protective layer, an excellent wear resistance and effect of suppressing corrosion of a magnetic recording medium are obtained, thus leading to realization of the present invention.

Hereinafter, preferable examples of a fluorine-containing ether compound, a lubricant for a magnetic recording medium (hereinafter, abbreviated as a “lubricant” in some cases), and a magnetic recording medium of the present invention will be described in detail. The present invention is not limited to only the embodiment shown below. Numbers, amount, positions, ratios, materials, configurations, and the like in the present invention can be added, omitted, replaced, or modified within the scope not departing from the gist of the present invention.

### [Fluorine-Containing Ether Compound]

A fluorine-containing ether compound of the present embodiment is represented by Formula (1) below.

R¹—CH₂—R²—CH₂—R³—CH₂—R⁴—CH₂—R⁵—CH₂—R⁶—CH₂—R⁷ (1)

(In Formula (1), R², R⁴, and R⁶ are perfluoropolyether chains having the same structure. R³ and R⁵ are each independently a linking group containing one or more hydroxyl groups, and R¹ and R⁷ are each independently a terminal group represented by Formula (2) below.)

—O—CH₂—CH(OH)—([D]—CH(OH))ₛ-[E]-CH₂OH (2)

(In Formula (2), s is 0 or 1, [D] and [E] each independently is a chain structure consisting of a combination of two to five methylene groups (—CH₂—) and one oxygen atom (—O—) or a chain structure consisting of one to four methylene groups (—CH₂—), provided that, in a case where s is 0 and [E] contains an oxygen atom, the number of methylene groups contained in [E] is 3 or more.)

(R¹ and R⁷)

In the fluorine-containing ether compound represented by Formula (1) above. R¹ and R⁷ are each independently a terminal group represented by Formula (2). A terminal group represented by Formula (2) contains two or three hydroxyl groups. Specifically, two hydroxyl groups are included when s in the terminal group represented by Formula (2) is 0, and three hydroxyl groups are included when s is 1. In the fluorine-containing ether compound represented by Formula (1), since R¹ and R⁷ each independently have two or three hydroxyl groups, in a case where a lubricating layer is formed on a protective layer using a lubricant containing the fluorine-containing ether compound, a suitable interaction is generated between the lubricating layer and the protective layer.

In the fluorine-containing ether compound represented by Formula (1), the total number of hydroxyl groups in R¹ and hydroxyl groups in R⁷ is 4 to 6. Since the above-described total number is 4 or more, the lubricating layer containing the fluorine-containing ether compound has high adhesiveness (adhesion properties) with respect to the protective layer. In addition, since the above-described total number is 6 or less, it is possible to prevent pickup which is adhesion of the fluorine-containing ether compound to a magnetic head as foreign matter (smears) due to excessively high polarity in a magnetic recording medium having the lubricating layer containing the fluorine-containing ether compound.

The number of hydroxyl groups in R¹ is preferably the same as the number of hydroxyl groups in R³. That is, it is preferable that R¹ and R⁷ each independently contain two hydroxyl groups (for example, R¹ and R⁷ are represented by any of Formulae (2-1) to (2-3) below) or R¹ and R⁷ each independently contain three hydroxyl groups (for example, R¹ and R⁷ are represented by any of Formulae (3-1) to (3-4) below). In this case, since the lubricant containing the fluorine-containing ether compound adheres closely to the protective layer with a good balance, a lubricating layer with a high coating rate is likely to be obtained. In particular, in the case where R¹ and R⁷ each independently contain three hydroxyl groups, the hydroxyl groups contained in the fluorine-containing ether compound can firmly adhere closely to the protective layer, and therefore the coating rate increases and the wear resistance is excellent.

Each hydroxyl group in R¹ and R⁷ is bound to a different carbon atom. In R¹ and R⁷, the carbon atoms to which a hydroxyl group is bound are bound to each other via a linking group containing carbon atoms to which no hydroxyl group is bound. For this reason, the fluorine-containing ether compound represented by Formula (1) has favorable hydrophobicity compared to, for example, a compound having terminal groups in which carbon atoms to which a hydroxyl group is bound are bound to each other. As a result, it is inferred that the lubricating layer containing the fluorine-containing ether compound represented by Formula (1) can prevent water from intruding and effectively suppress corrosion of a magnetic recording medium.

In addition, in the case where carbon atoms to which a hydroxyl group is bound are bound to each other, either the terminal hydroxyl group or the hydroxyl group adjacent to the terminal hydroxyl group is oriented in the opposite direction with respect to the protective layer. For this reason, either the terminal hydroxyl group or the hydroxyl group adjacent to the terminal hydroxyl group is less likely to adhere closely to the protective layer.

On the other hand, in the case where carbon atoms to which a hydroxyl group is bound are bound to each other via a linking group containing carbon atoms to which no hydroxyl group is bound, the linking group containing the carbon atoms to which no hydroxyl group is bound allows both the terminal hydroxyl group and the hydroxyl group adjacent to the terminal hydroxyl group to be oriented so as to adhere closely to the protective layer. From this, it is inferred that, in the case where carbon atoms to which a hydroxyl group is bound are bound to each other via a linking group containing carbon atoms to which no hydroxyl group is bound, an excellent wear resistance would be obtained.

In the terminal group represented by Formula (2), a linear linking group having an appropriate number of carbon atoms is placed between a carbon atom to which a hydroxyl group is bound and a carbon atom to which a hydroxyl group adjacent to the hydroxyl group is bound. Accordingly, the distance between the hydroxyl groups contained in the terminal group represented by Formula (2) is appropriate. Furthermore, in each terminal group represented by Formula (2), even in a case where a linking group placed between a carbon atom to which the most terminal hydroxyl group (terminal hydroxyl group) is bound and a carbon atom to which a hydroxyl group adjacent to the terminal hydroxyl group is bound contains an oxygen atom, the number of methylene groups contained in the above-described linking group is appropriate. Therefore, the lubricating layer containing the fluorine-containing ether compound of the present embodiment has appropriate hydrophobicity and favorable adhesion properties with respect to the protective layer. As a result, an excellent wear resistance and effect of suppressing corrosion of a magnetic recording medium are obtained.

If the distance between the hydroxyl groups contained in the terminal group represented by Formula (2) is too close, intramolecular aggregation between the hydroxyl groups is likely to occur. However, if the distance between the hydroxyl groups contained in the terminal group represented by Formula (2) is too far, the hydrophobicity becomes too high and the adhesion properties with respect to the protective layer become insufficient.

In addition, in a case where a linking group placed between a carbon atom to which a terminal hydroxyl group is bound and a carbon atom to which a hydroxyl group adjacent to the terminal hydroxyl group is bound contains an oxygen atom (ether bond), if the number of methylene groups contained in the above-described linking group is too small, intramolecular aggregation between the terminal hydroxyl group and the hydroxyl group adjacent thereto is likely to occur due to flexibility resulting from the ether bond. In addition, if the number of methylene groups contained in the above-described linking group is too large, the hydrophobicity becomes too high and the adhesion properties with respect to the protective layer become insufficient.

In Formula (2). [D] and [E] each independently is a chain structure consisting of a combination of two to five methylene groups (—CH₂—) and one oxygen atom (—O—) or a chain structure consisting of one to four methylene groups (—CH₂—). Examples of the chain structure consisting of a combination of two to five methylene groups and one oxygen atom include, if the left side is regarded as the perfluoropolyether side and the right side is regarded as the terminal side, —CH₂—O—CH₂, —CH₂—O—CH₂CH₂—, —CH₂O—CH₂CH₂CH₂—, —CH₂CH₂—O—CH₂—, —CH₂CH₂—O—CH₂—CH₂—, —CH₂CH₂—O—CH₂CH₂CH₂—, —CH₂CH₂CH₂—O—CH₂—, and —CH₂CH₂CH₂—O—CH₂CH₂—.

To obtain moderate flexibility due to inclusion of the ether bond, [D] preferably is a chain structure consisting of a combination of two to five methylene groups and one oxygen atom and more preferably is a chain structure consisting of a combination of two to four methylene groups and one oxygen atom.

In Formula (2), in a case where s is 0 and [E] contains an oxygen atom, the number of methylene groups contained in [E] is 3 or more. That is, in a case where [E] contains an oxygen atom, [B] is a chain structure consisting of a combination of three to five methylene groups (—CH₂—) and one oxygen atom (—O—).

In the fluorine-containing ether compound represented by Formula [1], the terminal group represented by Formula (2) is preferably a terminal group represented by any of Formulae (2-1) to (2-3) and (3-1) to (3-4) below. In this case, a lubricating layer having an excellent wear resistance and excellent effect of suppressing corrosion of a magnetic recording medium can be formed.

Furthermore, at least one of R¹ and R⁷ in Formula (1) is preferably the terminal group represented by any of Formula (3-1) to (3-4). In a case where at least one of R¹ and R⁷ is the terminal group represented by any of Formula (3-1) to (3-4), a fluorine-containing ether compound in which R¹ and/or R⁷ contain three hydroxyl groups is obtained. As a result, a lubricating layer having superior adhesion properties with respect to the protective layer and a more favorable wear resistance can be formed.

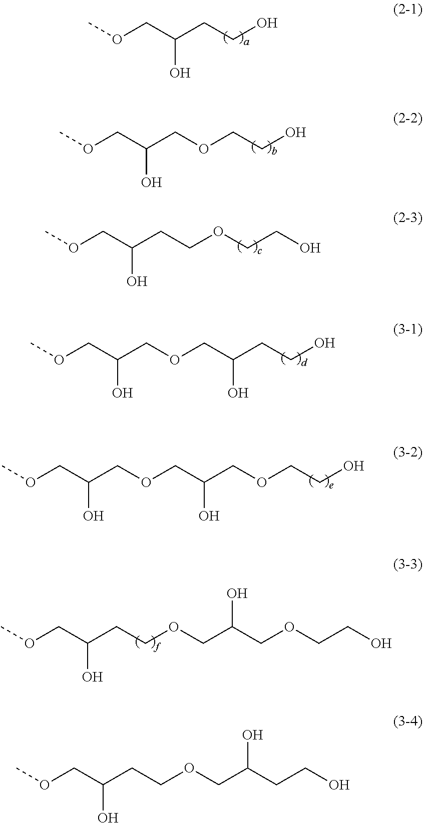

(a in Formula 12-1) is an integer of 1 to 4.)
(b in Formula (2-2) is an integer of 2 to 3.)
(e in Formula (2-3) is an integer of 1 to 3.)
(d in Formula (3-1) is an integer of 1 to 4.)
(e in Formula (3-23 is an integer of 1 to 3.)
(f in Formula (3-3) is an integer of 1 to 2.)

The terminal group represented by Formula (2-1) above has two hydroxyl groups. The terminal groups represented by Formulae (3-1) and (3-4) above each independently have three hydroxyl groups. In all the terminal groups represented by Formulae (2-1), (3-1), and (3-4) above, each linking group between a carbon atom to which a terminal hydroxyl group is bound and a carbon atom to which a hydroxyl group adjacent to the terminal hydroxyl group is bound does not contain an oxygen atom. The above-described linking groups in the terminal groups represented by Formulae (2-1), (3-1), and (3-4) above all have a linear structure containing 1 to 4 carbon atoms to which no hydroxyl group is bound. In a case where the above described linking groups do not contain oxygen atoms and have a linear structure containing 1 or more carbon atoms to which no hydroxyl group is bound, a fluorine-containing ether compound having favorable hydrophobicity is obtained. In addition, if the above-described linking groups have a linear structure containing 4 or less carbon atoms, problems are not caused in adhesion properties with respect to the protective layer due to too hydrophobic linking groups. In the terminal groups represented by Formulae (2-1), (3-1), and (3-4) above, each linking group between a carbon atom to which a terminal hydroxyl group is bound and a carbon atom to which a hydroxyl group adjacent to the terminal hydroxyl group is bound does not contain an oxygen atom. For this reason, intramolecular interaction is small, intramolecular aggregation is less likely to occur, and a lubricating layer having excellent adhesion properties with respect to the protective layer is obtained. Accordingly, a lubricating layer containing a fluorine-containing ether compound in which the above-described linking groups have a linear structure containing the number of carbon atoms within the above-described range exhibits a high wear resistance, can prevent water from intruding, and is highly effective in suppressing corrosion of a magnetic recording medium.

The terminal groups represented by Formulae (2-2) and (2-3) above each independently have two hydroxyl groups. In the terminal groups represented by Formulae (2-2) and (2-3) above, each linking group placed between a carbon atom to which a terminal hydroxyl group is bound and a carbon atom to which a hydroxyl group adjacent to the terminal hydroxyl group is bound contains an oxygen atom. The above-described linking groups have a linear structure containing 3 to 5 carbon atoms to which no hydroxyl group is bound. Even in the case where the above-described linking groups contain an oxygen atom, if the linking groups have a linear structure containing 3 or more carbon atoms to which no hydroxyl group is bound, a fluorine-containing ether compound having favorable hydrophobicity is obtained. In addition, if the above-described linking groups have a linear structure containing 5 or less carbon atoms, problems are not caused in adhesion properties with respect to the protective layer due to too hydrophobic linking groups. Accordingly, a lubricating layer containing a fluorine-containing ether compound in which the above-described linking groups have a linear structure containing the number of carbon atoms within the above-described range exhibits a high wear resistance, can prevent water from intruding, and is highly effective in suppressing corrosion of a magnetic recording medium.

In addition, in the terminal groups represented by Formulae (2-2) and (2-3), the above-described linking groups have a linear structure consisting of 4 to 6 atoms. If the number of atoms contained in each of the abo escribed linking groups is within the above-described range, even if the above described linking groups contain an oxygen atom, the molecular mobility is appropriate, the intramolecular aggregation is less likely to occur, and a lubricating layer having excellent adhesion properties with respect to the protective layer is obtained.

The terminal groups represented by Formulae (3-2) and (3-3) above each independently have three hydroxyl groups. In the terminal groups represented by Formulae (3-2) and (3-3) above, each linking group placed between a carbon atom to which a terminal hydroxyl group is bound and a carbon atom to which a hydroxyl group adjacent to the terminal hydroxyl group is bound contains an oxygen atom. The above-described linking groups have a linear structure containing 2 to 4 carbon atoms to which no hydroxyl group is bound. Even in the case where the above-described linking groups contain an oxygen atom, if the linking groups have a linear structure containing 2 or more carbon atoms to which no hydroxyl groups are bound, a fluorine-containing ether compound having favorable hydrophobicity is obtained. In addition, if the above-described linking groups have a linear structure containing 4 or less carbon atoms, problems are not caused in adhesion properties with respect to the protective layer due to too hydrophobic linking groups. In addition, even if the above-described linking groups contain an oxygen atom, since the terminal groups represented by Formulae (3-2) and (3-3) have three hydroxyl groups, if the linking groups have a linear structure containing 2 or more carbon atoms to which no hydroxyl group is bound, a lubricating layer having excellent adhesion properties with respect to the protective layer is obtained. Accordingly, a lubricating layer containing a fluorine-containing ether compound in which the above-described linking groups have a linear structure containing the number of carbon atoms within the above-described range exhibits a high wear resistance, can prevent water from intruding, and is highly effective in suppressing corrosion of a magnetic recording medium.

In addition, in the terminal groups represented by formulae (3-2) and (3-3), the above-described linking groups have a linear structure consisting of 3 to 5 atoms. If the number of atoms contained in each of the above-described linking groups is within the above-described range, even if the above-described linking groups contain an oxygen atom, the molecular mobility is appropriate, the intramolecular aggregation is less likely to occur, and a lubricating layer having excellent adhesion properties with respect to the protective layer is obtained.

In the terminal group represented by Formula (2-1), s in Formula (2) is 0 and [E] is a chain structure consisting of one to four methylene groups. In the terminal group represented by Formula (2-1), a is an integer of 1 to 4. Since a linking group between a carbon atom to which a terminal hydroxyl group is bound and a carbon atom to which a hydroxyl group adjacent to the terminal hydroxyl group is bound does not contain an oxygen atom, appropriate hydrophobicity is exhibited. In addition, since a is 4 or less, problems are not caused in adhesion properties with respect to the protective layer due to too hydrophobic linking groups, thereby exhibiting an excellent effect of suppressing corrosion of a magnetic recording medium. Furthermore, since the linking group between a carbon atom to which a terminal hydroxyl group is bound and a carbon atom to which a hydroxyl group adjacent to the terminal hydroxyl group is bound does not contain an oxygen atom, intramolecular interaction between the above-described hydroxyl groups is small. For this reason, a fluorine-containing ether compound having the terminal group represented by Formula (2-1) is less likely to cause intramolecular aggregation and can form a lubricating layer having excellent adhesion properties with respect to the protective layer and an excellent wear resistance.

In the terminal group represented by Formula (2-2), s in Formula (2) is 0 and [E] is a chain structure consisting of a combination of three or four methylene groups and one oxygen atom. In the terminal group represented by Formula (2-2), b is an integer of 2 to 3. For this reason, a fluorine-containing ether compound having favorable hydrophobicity is obtained, and an excellent effect of suppressing corrosion of a magnetic recording medium is exhibited. In the terminal group represented by Formula (2-2), a linking group between a carbon atom to which a terminal hydroxyl group is bound and a carbon atom to which a hydroxyl group adjacent to the terminal hydroxyl group is bound contains an oxygen atom. However, in the terminal group represented by Formula (2-2), since b is 2 to 3, the molecular mobility is appropriate, the intramolecular aggregation between the hydroxyl groups is less likely to occur, and a lubricating layer having excellent adhesion properties with respect to the protective layer and an excellent wear resistance can be formed.

In the terminal group represented by Formula (2-3), s in Formula (2) is 0 and [E] is a chain structure consisting of a combination of three to five methylene groups and one oxygen atom. In the terminal group represented by Formula (2-3), e is an integer of 1 to 3. For this reason, a fluorine-containing ether compound having favorable hydrophobicity is obtained, and an excellent effect of suppressing corrosion of a magnetic recording medium is exhibited. In the terminal group represented by Formula (2-3), a linking group between a carbon atom to which a terminal hydroxyl group is bound and a carbon atom to which a hydroxyl group adjacent to the terminal hydroxyl group is bound contains an oxygen atom. However, in the terminal group represented by Formula (2-3), since c is 1 to 3, the molecular mobility is appropriate, the intramolecular aggregation between the above-described hydroxyl groups is less likely to occur, and a lubricating layer having excellent adhesion properties with respect to the protective layer and an excellent wear resistance can be formed.

In the terminal group represented by Formula (3-1), s in Formula (2) is 1, [D] is a chain structure consisting of a combination of two methylene groups and one oxygen atom, and [E] is a chain structure consisting of one to four methylene groups. In the terminal group represented by Formula (3-1), d is an integer of 1 to 4. Since a linking group between a carbon atom to which a terminal hydroxyl group is bound and a carbon atom to which a hydroxyl group adjacent to the terminal hydroxyl group is bound does not contain an oxygen atom, appropriate hydrophobicity is exhibited. In addition, since dis 4 or less, problems are not caused in adhesion properties with respect to the protective layer due to too hydrophobic linking groups, thereby exhibiting an excellent effect of suppressing corrosion of a magnetic recording medium. Furthermore, in the terminal group represented by Formula (3-1), since a linking group between a carbon atom to which a terminal hydroxyl group is bound and a carbon atom to which a hydroxyl group adjacent to the terminal hydroxyl group is bound does not contain an oxygen atom and the terminal group contains three hydroxyl groups, a lubricating layer having excellent adhesion properties with respect to the protective layer and an excellent wear resistance can be formed.

In the terminal group represented by Formula (3-2), s in Formula (2) is 1. [D] is a chain structure consisting of a combination of two methylene groups and one oxygen atom, and [E] is a chain structure consisting of a combination of two to four methylene groups and one oxygen atom. In the terminal group represented by Formula (3-2), e is an integer of 1 to 3. For this reason, a fluorine-containing ether compound having favorable hydrophobicity is obtained, and an excellent effect of suppressing corrosion of a magnetic recording medium is exhibited. In addition, the terminal group represented by Formula (3-2) contains three hydroxyl groups, and therefore a lubricating layer having excellent adhesion properties with respect to the protective layer and an excellent wear resistance can be formed.

In the terminal group represented by Formula (3-3), s in Formula (2) is 1. [D] is a chain structure consisting of a combination of three or four methylene groups and one oxygen atom, and [E] is a chain structure consisting of a combination of two methylene groups and one oxygen atom. In the terminal group represented by Formula (3-3), f is an integer of 1 to 2. For this reason, a fluorine-containing ether compound having favorable hydrophobicity is obtained, and an excellent effect of suppressing corrosion of a magnetic recording medium is exhibited. In addition, the terminal group represented by Formula (3-3) contains three hydroxyl groups, and therefore a lubricating layer having excellent adhesion properties with respect to the protective layer and an excellent wear resistance can be formed.

In the terminal group represented by Formula (3-4), s in Formula (2) is 1, [D] is a chain structure consisting of a combination of three methylene groups and one oxygen atom, and [E] is a chain structure consisting of one methylene group. In the terminal group represented by Formula (3-4), a linking group between a carbon atom to which a terminal hydroxyl group is bound and a carbon atom to which a hydroxyl group adjacent to the terminal hydroxyl group is bound does not contain an oxygen atom. For this reason, a fluorine-containing ether compound having favorable hydrophobicity is obtained, and an excellent effect of suppressing corrosion of a magnetic recording medium is exhibited. In addition, the terminal group represented by Formula (3-4) contains three hydroxyl groups, and therefore a lubricating layer having excellent adhesion properties with respect to the protective layer and an excellent wear resistance can be formed.

In the fluorine-containing ether compound represented by Formula (1). R¹ and R⁷ may be the same as of different from each other, and are preferably the same as each other. If R¹ and R⁷ are the same as each other, a fluorine-containing ether compound which is likely to wet and spread evenly on the protective layer and from which a lubricating layer having a uniform film thickness is likely to be obtained is obtained.

As a result, the lubricating layer containing this fluorine-containing ether compound has a favorable coating rate and an excellent wear resistance, which is preferable. In addition, in the case where R¹ and R⁷ are the same as each other, a fluorine-containing ether compound can be produced efficiently through fewer production steps compared to a case where R¹ and R⁷ are different from each other.

(R³ and R⁵)

In the fluorine-containing ether compound represented by Formula (1), R³ and R⁵ are each independently a linking group having one or more hydroxyl groups. The hydroxyl groups in R³ and R⁵ improve adhesion properties of the lubricating layer containing the fluorine-containing ether compound with respect to the protective layer. The number of hydroxyl groups contained in each of R³ and R⁵ is preferably 1 to 4 and more preferably 1 or 2 to maintain an appropriate coating rate, and still more preferably 1 for appropriate molecular hydrophobicity.

R³ and R⁵ are each a divalent linking group bound to methylene groups (—CH₂—) arranged on both sides. R³ and R⁵ are each preferably an alkylene group having one or more hydroxyl groups and containing an ether bond (—O—). The number of carbon atoms contained in each of R³ and R⁵ is preferably 3 to 12, more preferably 3 to 6, and still more preferably 3. Specifically, R³ and R⁵ are preferably linking groups represented by Formula (4) below.

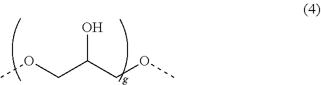

(g in Formula (4) is an integer of 1 to 4.)

In the linking group represented by Formula (4), g is an integer of 1 to 4. The hydroxyl groups in the linking group represented by Formula (4) improve adhesion properties of the lubricating layer containing the fluorine-containing ether compound with respect to the protective layer. In a case where g is 1, a fluorine-containing ether compound is easily synthesized, which is preferable.

In addition, in the case where R³ and R⁵ are linking groups represented by Formula (4), oxygen atoms placed at both end portions of each of R³ and R⁵ are respectively bound to methylene groups (—CH₂—) placed on both sides of each of the linking groups represented by Formula (4) to form ether bonds (—O—). These four ether bonds impart moderate flexibility to the fluorine-containing ether compound represented by Formula (1) and increase the affinity between the protective layer and the hydroxyl groups in R³ and R⁵.

(R², R⁴, and R⁶)

R², R⁴, and R⁶ in the fluorine-containing ether compound represented by Formula (1) above are perfluoropolyether chains (PFPE chains) having the same Structure. Due to the PPPE chains represented by R², R⁴, and R⁶, in a case where a lubricant containing the fluorine-containing ether compound of the present embodiment is applied onto a protective layer to form a lubricating layer, the surface of the protective layer is covered and lubricity is imparted to the lubricating layer to reduce frictional force between a magnetic head and the protective layer. In addition, since the PFPE chains have low surface energy, water resistance is imparted to the lubricating layer containing the fluorine-containing ether compound of the present embodiment and the corrosion resistance of the magnetic recording medium on which the lubricating layer is provided is improved.

R², R⁴, and R⁶ in Formula (1) are PFPE chains having the same structure and can be appropriately selected depending on the performance and the like required of a lubricant containing a fluorine-containing ether compound. Examples of PEPE chains include PEPE chains consisting of a perfluoromethylene oxide polymer, a perfluoroethylene oxide polymer, a perfluoro-n-propylene oxide polymer, a perfluoroisopropylene oxide polymer, and copolymers thereof.

The PFPE chains may have a structure represented by Formula (Rf) below derived from a perfluoroalkylene oxide polymer or copolymer, for example.

—(CF₂)_{w1}O(CF₂O)_{w2}(CF₂CF₂O)_{w3}(CF₂CF₂CF₂O)_{w4}(CF₂CF₂CF₂CF₂O)_{w5}(CF₂)_{w6}— (Rf)

(In Formula (Rf), w2, w3, w4, and w5 indicate the average degree of polymerization and each independently represent 0 to 20, provided that all of w2, w3, w4, and w5 are not 0 at the same time, w1 and w6 are average values indicating the number of —CF₂— and each independently represent 1 to 3, and the arrangement sequence of repeating units in Formula (Rf) is not particularly limited.)

In Formula (Rf), w2, w3, w4, and w5 indicate the average degree of polymerization and each independently represent 0 to 20, preferably 0 to 15, more preferably 0 to 10.

In Formula (Rf), w1 and w6 are average values indicating the number of —CF₂— and each independently represent 1 to 3, w1 and w6 are determined according to the structure or the like of the repeating units arranged at the end portions of the chain structure in the polymer represented by Formula (Rf).

(CF₂O), (CF₂CF₂O). (CF₂CF₂CF₂O), and (CF₂CF₂CF₂CF₂O) in Formula (Rf) are repeating units. The arrangement sequence of the repeating units in Formula (Rf) are not particularly limited. In addition, the number of types of repeating units in Formula (Rf) are not particularly limited.

It is preferable that R², R⁴, and R⁶ in Formula (1) be, for example, PFPE chains represented by Formula (Rf-1) below.

(CF₂)_{w7}O—(CF₂CF₂O)_{w8}—(CF₂CF₂CF₂O)_{w9}—(CF₂)_{w10}— (Rf-1)

(In Formula (Rf-1), w8 and w9 indicate the average degree of polymerization and each independently represent 0.1 to 20, and w7 and w10 are average values indicating the number of —CF₂— and each independently represent 1 to 2.)

The arrangement sequence of (CF₂CF₂O)) and (CF₂CF₂CF₂O)) which are repeating units in Formula (Rf-1) is not particularly limited. Formula (Rf-1) may include any of a random copolymer, a block copolymer, and an alternating copolymer composed of the monomer units (CF₂CF₂O) and (CF₂CF₂CF₂O)). In Formula (Rf-1), w8 and w9 indicate the average degree of polymerization and each independently represent 0.1 to 20, preferably 0.1 to 15, more preferably 1 to 10. In Formula (Rf-1), w7 and w10 are average values indicating the number of —CF₂— and each independently represent 1 to 2, w7 and w10 are determined according to the structure or the like of the repeating units arranged at the end portions of the chain structure in the polymer represented by Formula (Rf-1).

It is also preferable that R², R⁴, and R⁶ in Formula (1) be any of Formulae (5) to (9) below. The arrangement sequence of (CF₂CF₂O) and (CF₂O)) which are repeating units in Formula (S) is not particularly limited. Formula (5) may include any of a random copolymer, a block copolymer, and an alternating copolymer composed of the monomer units (CF₂—CF₂—O) and (CF₂—O).

—CF₂O—(CF₂CF₂O)ₕ—(CF₂O)ᵢ—CF₂— (5)

(In Formula (5), h and i indicate the average degree of polymerization and each independently represent 0.1 to 20.)

—CF₂O—(CF₂CF₂O)ⱼ—CF₂— (6)

(In Formula (6), j indicates the average degree of polymerization and represents 0.1 to 20.)

—CF₂CF₂O—(CF₂CF₂CF₂O)ₖ—CF₂CF₂— (7)

(In Formula (7), k indicates the average degree of polymerization and represents 0.1 to 20.)

—CF₂CF₂CF₂O—(CF₂CF₂CF₂CF₂O)ₗ—CF₂CF₂CF₂— (8)

(In Formula (8), 1 indicates the average degree of polymerization and represents 0.1 to 10.)

—CF(CF₃)O—(CF₂CF(CF₃)O)ᵣ—CF(CF₃)— (9)

(In Formula (9), r indicates the average degree of polymerization and represents 0.1 to 20.)

h and i indicating average degrees of polymerization in Formula (5) are each 0.1 to 20, j indicating the average degree of polymerization in Formula (6) is 0.1 to 20, k indicating the average degree of polymerization in Formula (7) is 0.1 to 20, 1 indicating the average degree of polymerization in Formula (8) is 0.1 to 10, and r indicating the average degree of polymerization in Formula (9) is 0.1 to 20, h, i, j, k, l, and r can be arbitrarily selected within the above-described ranges. If h, i, j, k, l, and r indicating average degrees of polymerization are each 0.1 or more, a fluorine-containing ether compound from which a lubricating layer having a favorable wear resistance and capable of suppressing corrosion of a magnetic recording medium can be obtained is obtained. In addition, if h, i, j, k, and r indicating average degrees of polymerization are each 20 or less and 1 is 10 or less, the viscosity of a fluorine-containing ether compound does not become too high, and a lubricant containing this fluorine-containing ether compound becomes easy to apply, which is preferable. All of b, i, j, k, l, and r indicating the average degree of polymerization are preferably 1 to 10 and more preferably 2 to 8 to obtain a fluorine-containing ether compound which is likely to wet and spread on a protective layer and from which a lubricating layer having a uniform film thickness is likely to be obtained h, i, j, k, l, and r may be, for example, 0.5 to 9, 1 to 8, 2 to 7, 3 to 6, or 4 to 5.

In the case where R², R⁴, and R⁶ in Formula (1) is any of Formulae (5) to (9), a fluorine-containing ether compound is easily synthesized, which is preferable. In a case where R², R⁴, and R⁶ are any of Formulae (5) to (7), a raw material is readily available, which is preferable. In addition, in the case where R², R⁴, and R⁶ are any of Formulae (5) to (9), the ratio of the number of oxygen atoms (the number of ether bonds (—O—) to the number of carbon atoms in the perfluoropolyether chains is appropriate. For this reason, a fluorine-containing ether compound with moderate hardness is obtained. Accordingly, a fluorine-containing ether compound applied onto a protective layer is less likely to be aggregated on the protective layer, and a lubricating layer having an even thinner thickness at a sufficient coating rate can be formed.

R², R⁴, and R⁶ in the fluorine-containing ether compound represented by Formula (1) are PEPE chains having the same structure. In the present specification, PFPE chains having the same structure include a case where PFPE chains have the same Structure (repeating unit) but have different average degrees of polymerization. For example, in a case where R², R⁴, and R⁶ are all PFPE chains represented by Formula (5). R², R⁴, and R⁶ all have the same repeating units ((CF₂CF₂O) and (CF₂O)), and h and i indicating average degrees of polymerization in Formula (5) may be partially or entirely the same in R², R⁴, and R⁶ or may be all different. If R², R⁴, and R⁶ are PEPE chains having the same structure and at least the PFPB chains of R² and R⁶ have the same average degrees of polymerization, a fluorine-containing ether compound is easily synthesized, which is preferable. R², R⁴, and R⁶ may be PFPE chains having the same structure and all the PEPE chains of R², R⁴, and R⁶ may have the same average degrees of polymerization.

In the case where R², R⁴, and R⁶ are PPPE chains having the same structure, a fluorine-containing ether compound having less molecular strain due to the mobility of the PFPE chains is obtained. Accordingly, a lubricating layer that can adhere closely and uniformly to the protective layer and has a high coating rate can be formed.

On the other hand, in a case of a fluorine-containing ether compound in which PEPE chains having different structures are mixed in its molecule (for example, in a case where R² and R⁶ are PFPE chains represented by Formula (5), and R⁴ is a PFPE chain represented by Formula (6)), molecular strain occurs due to the difference in mobility caused by the structures of the PEPE chains, whereby the molecular linearity deteriorates. Accordingly, it is inferred that a lubricating layer that cannot adhere closely and uniformly to the protective layer and has a low coating rate would be formed.

R², R⁴, and R⁶ in the fluorine-containing ether compound represented by Formula (1) have the same structure. Furthermore, in the fluorine-containing ether compound represented by Formula (1), it is preferable that R³ and R⁵ be the same as each other and R¹ and R⁷ be the same as each other. Such a fluorine-containing ether compound has a symmetrical structure around R⁴. For this reason, the fluorine-containing ether compound is likely to wet and spread on the protective layer and a lubricating layer having a more uniform film thickness is likely to be obtained. In addition, a fluorine-containing ether compound in which R², R⁴, and R⁶ are the same as each other, R³ and R⁵ are also the same as each other, and R¹ and R⁷ are also the same as each other can be produced easily and efficiently through fewer production steps.

It is preferable that the fluorine-containing ether compound represented by Formula (1) be specifically any compound represented by Formulae (A) to (M) below.

Since mc, mg, mb, nc, ng, nb, pa, pd, pi, pj, qb, qe, qf, qk, ql, and qm in Formulae (A) to (M) are values indicating average degrees of polymerization, these are not necessarily integers.

In all the compounds represented by Formulae (A) to (M) below, R¹ and R⁷ are the same as each other. In all the compounds represented by Formulae (A) to (M) below. R³ and R⁵ are the same as each other, and g in Formula (4) is 1. In all the compounds represented by Formulae (A) to (M) below, R², R⁴, and R⁶ are the same as each other.

In the compound represented by Formula (A) below, R¹ and R⁷ are terminal groups represented by Formula (2-1), a is 2, and R², R⁴, and R⁶ are PFPE chains represented by Formula (6).

In the compound represented by Formula (B) below, R¹ and R⁷ are terminal groups represented by Formula (2-1), a is 3, and R², R⁴, and R⁶ are PFPE chains represented by Formula (7).

In the compound represented by Formula (C) below, R¹ and R⁷ are terminal groups represented by Formula (2-2), b is 2, and R², R⁴, and R⁶ are PEPE chains represented by Formula (S).

In the compound represented by Formula (D) below, R¹ and R⁷ are terminal groups represented by Formula (2-2), b is 3, and R³, R⁴, and R⁶ are PFPE chains represented by Formula (6).

In the compound represented by Formula (E) below, R¹ and R⁷ are terminal groups represented by Formula (3-2), e is 1, and R², R⁴, and R⁶ are PEPE chains represented by Formula (7).

In the compound represented by Formula (F) below, R¹ and R⁷ are terminal groups represented by Formula (3-2), e is 2, and R², R⁴, and R⁶ are PFPE chains represented by Formula (7).

In the compound represented by Formula (G) below, R¹ and R⁷ are terminal groups represented by Formula (2-3), c is 1, and R², R⁴, and R⁶ are PFPE chains represented by Formula (5).

In the compound represented by Formula (H) below, R¹ and R⁷ are terminal groups represented by Formula (3-3), f is 1, and R², R⁴, and R⁶ are PFPE chains represented by Formula (5.

In the compound represented by Formula (I) below. R¹ and R⁷ are terminal groups represented by Formula (3-1), d is 1, and R², R⁴, and R⁶ are PFPE chains represented by Formula (6).

In the compound represented by Formula (J) below. R¹ and R⁷ are terminal groups represented by Formula (3-1), d is 2, and R², R⁴, and R⁶ are PEPE chains represented by Formula (6).

In the compound represented by Formula (K) below, R¹ and R⁷ are terminal groups represented by Formula (3-3), f is 2, and R², R⁴, and R⁶ are PFPB chains represented by Formula (7).

In the compound represented by Formula (L) below, R¹ and R⁷ are terminal groups represented by Formula (3-4), and R², R⁴, and R⁶ are PFPE chains represented by Formula (7).

In the compound represented by Formula (M) below, R¹ and R⁷ are terminal groups represented by Formula (2-1), a is 1, and R², R⁴, and R⁶ are PFPE chains represented by Formula (7).

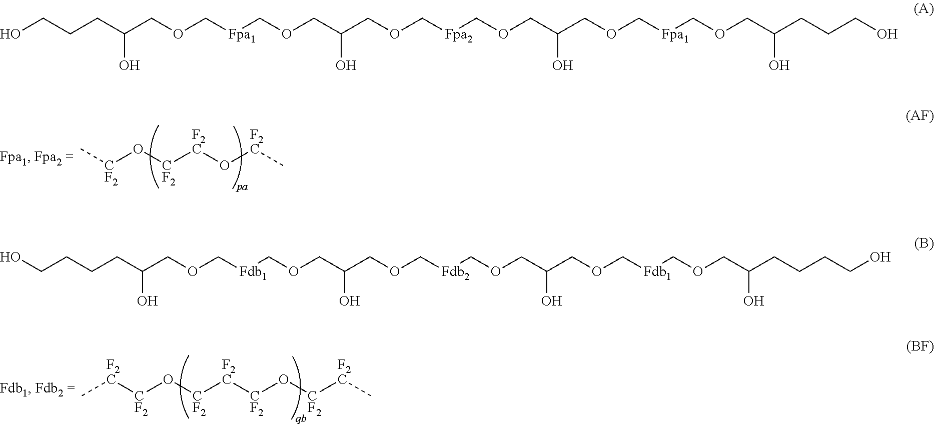

(Fpa₁ and Fpa₂) in Formula (A) are represented by Formula (AF), pa in Fpa₁ and Fpa₂ indicates the average degree of polymerization and resents 0.1 to 20, and pa in Fpa₁ and pa in Fpa₂ may be the sane as or different from each other.)

(Fdb₁ and Fdb₂ in Formula (B) are represented by Formula (BF), qb in Fdb₁ and Fdb₂ indicates the average degree of polymerization and represents 0.1 to 20, and qb in Fdb₁ and qb in Fdb₂ may be the same a, or different from each other.)

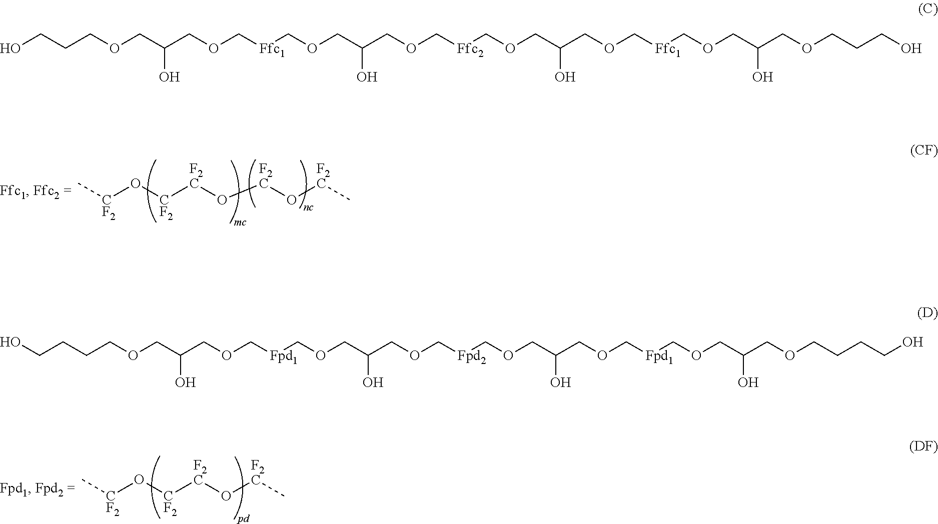

(Ffc₁ and Ffc₂ in Formula (C) are represented by Formula (CF), mc and nc in Ffc₁ and Ffc₂ each indicate the average degree of polymerization and represent 0.1 to 20, and mc and nc in Ffc₁ and me and nc in Ffc₂ may be the same as or different from each other.)

(Fpd₁ and Fpd₂ in Formula (D) are represented by Formula (DF), pd in Fpd₁ and Fpd₂ indicates the average degree of polymerization and represents 0.1 to 20, and pd in Fpd₁ and pd in Fpd₂ may be the same as or different from each other.)

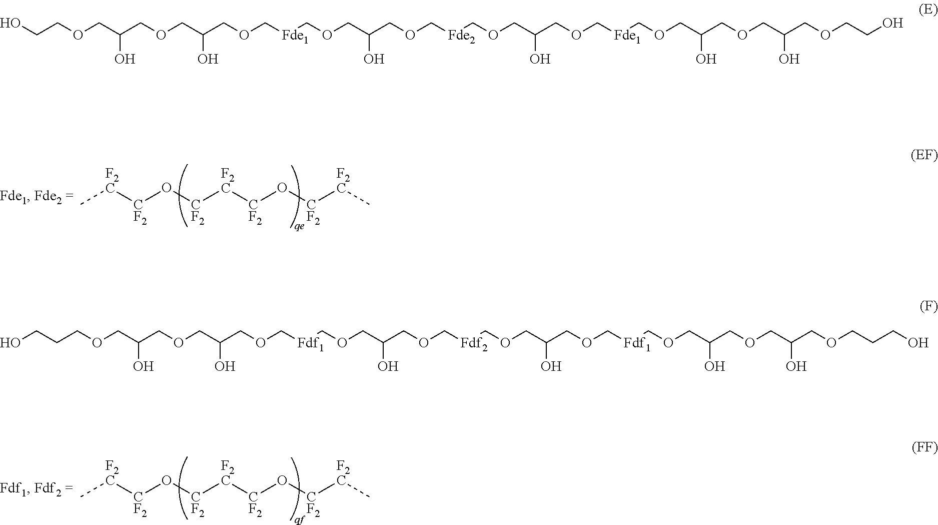

(Fde₁ and Fde₂) in Formula (E) are represented by Formula (EF), qe in Fde₁ and Fde₂ indicates the average degree of polymerization and represents 0.1 to 20, and qe in Fde₁ and qe in Fde₂ may be the same a or different from each other.)

(Fde₁ and Fde₂ in Formula (F) are represented by Formula (FF), qf in Fdf₁ and Fdf₂ indicates the average degree of polymerization and represents 0.1 to 20, and qf in Fdf₁ and qf in Fdf₂ may be the same as or different from each other.)

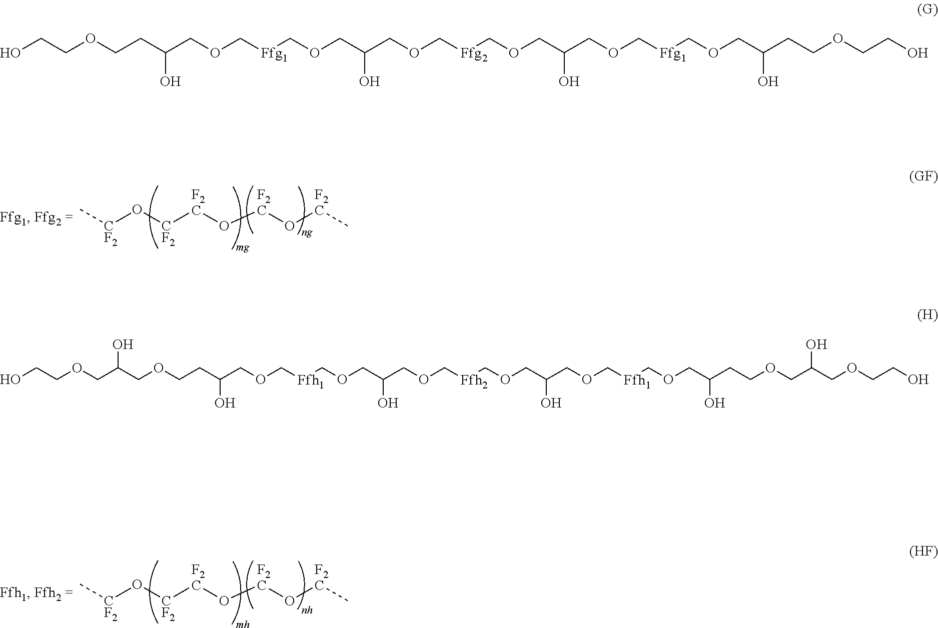

(Ffg₁ and Ffg₂ in Formula (G) are represented by Formula (GF), mg and ng in Ffg₁ and Ffg₂ each indicate the average degree of polymerization and represent 0.1 to 20, and mg and ng in Ffg₁ and mg and ng in Ffg₂ may be the same as or different from each other.)

(Ffh₁ and Ffh₂ in Formula (H) are represented by Formula (HF), mh and nh in Ffh₁, and Ffh₂ each indicate the average degree of polymerization and represent 0.1 to 20, and mh and nh in Ffh₁ and nh and nh in Fft₂ may be the same as or different from each other.)

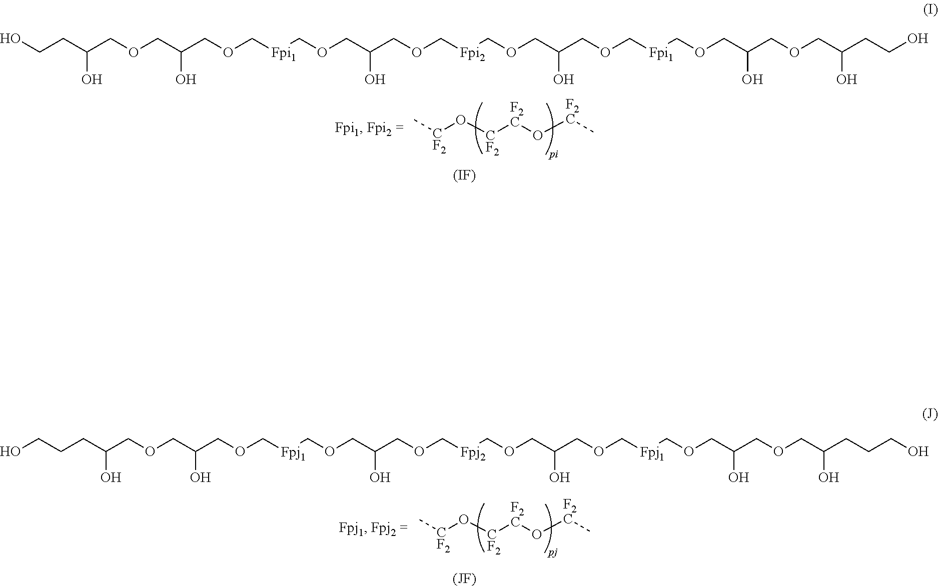

(Fpi₁ and Fpi₂ in Formula (1) are represented by Formula (1F), pi in Fpi₁ and Fpi₂ indicates the average degree of polymerization and represents 0.1 to 20, and pi in Fpi₁ and pi in Fpi₂ may be the same as or different from each other.)

(Fpj₁ and Fpj₂ in Formula (J) are represented by Formula (JF), pj in Fpj₁ and Fpj₂ indicate the average degree of polymerization and represents 0.1 to 20, and pj in Fpj₁ and pj in Fpj₂ may be the same as or different from each other.)

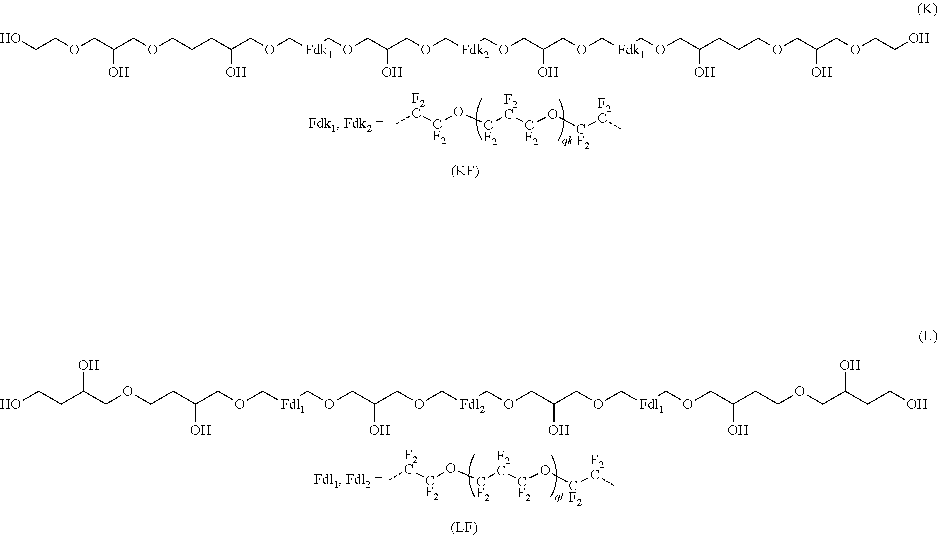

(Fdk₁ and Fdk₂ in Formula (K) are represented by Formula (KF), qk in Fdk₁ and Fdk₂ indicates the average degree of polymerization and represents 0.1 to 20, and qk in Fdk₁ and qk in Fdk₂ may be the same as or different from each other.)

(Fdl₁ and Fdl₂ in Formula (L) are represented by Formula (LF), ql in Fdl₁ and Fdl₂ indicates the average degree of polymerization and represents 0.1 to 20, and ql in Fdl₁ and ql in Fdl₂ may be the same as or different from each other.)

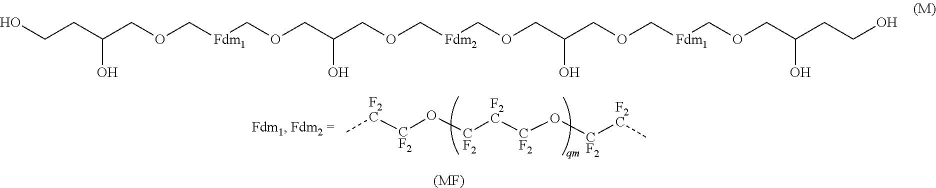

(Fdm₁ and Fdm₂ in Formula (M) are represented by Formula (MF), qui in Fdm₁ and Fdm₂ indicates the average degree of polymerization and represents 0.1 to 20, and qm in Fdm₁ and qm in Fdm₂ may be the same as or different from each other.)

When the compound represented by Formula (1) is any of compounds represented by Formulae (A) to (M), it is preferable because the procurement of raw materials is easy and a lubricating layer having an excellent wear resistance and capable of suppressing corrosion of a magnetic recording medium can be formed even if the lubricating layer has a thin thickness.

When the compound represented by Formula (1) is any of the compounds represented by Formulae (B) and (E) to (L), it is more preferable because it is possible to form a lubricating layer having a particularly excellent wear resistance and effect of suppressing corrosion of a magnetic recording medium.

In the fluorine-containing ether compound of the present embodiment, the number average molecular weight (Mn) of the compound is preferably within a range of 500 to 10,000, more preferably within a range of 700 to 7,000, and particularity preferably within a range of 1,000 to 5,000. If the number average molecular weight thereof is 500 or more, a lubricant containing the fluorine-containing ether compound of the present embodiment hardly evaporates, whereby the lubricant can be prevented from evaporating and transferring to a magnetic head. In addition, if the number average molecular weight is 10,000 or less, the fluorine-containing ether compound has an appropriate viscosity, and a thin lubricating layer can be easily formed by applying a lubricant containing this fluorine-containing ether compound. If the number average molecular weight is 5,000 or less, in a case where the fluorine-containing ether compound is applied to a lubricant, the viscosity of the lubricant becomes appropriate for handling, which is more preferable.

The number average molecular weight (Mn) of the fluorine-containing ether compound is a value measured by ¹H-NMR and ¹⁹F-NMR with AVANCE III400 manufactured by Bruker BioSpin Group. In the nuclear magnetic resonance (NMR) measurement, a sample is diluted with a single or mixed solvent of hexafluorobenzene, acetone-d, tetrahydrofuran-d, and the like and used in the measurement. As the reference of the ¹⁹F-NMR chemical shift, the peak of hexafluorobenzene was set to −164.7 ppm. As the reference of the ¹H-NMR chemical shift, the peak of acetone was set to 2.2 ppm.

### “Production Method”

A method for producing the fluorine-containing ether compound of the present embodiment is not particularly limited, and the fluorine-containing ether compound can be produced using a well-known conventional production method. The fluorine-containing ether compound of the present embodiment can be produced using, for example, a production method shown below.

In the present embodiment, a case of producing a compound in which three PEPE chains represented by R², R⁴, and R⁶ have the same structure, R¹ is the same as R⁷, and R³ is the same as R⁵ will be described as an example of the fluorine-containing ether compound represented by Formula (1).

First, a fluorine compound is prepared in which hydroxymethyl groups (—CH₂OH) are placed at both terminals of a perfluoropolyether chain corresponding to R⁴ in Formula (1). Next, the hydroxyl groups of the hydroxymethyl groups placed at both terminals of the fluorine compound are reacted with a halogen compound having an epoxy group corresponding to R³ (═R⁵) (first reaction). As a result, an intermediate compound 1 having epoxy groups corresponding to R³ (═R⁵) at both terminals of the perfluoropolyether chain corresponding to R⁴ is obtained.

Subsequently, a fluorine compound is prepared in which hydroxymethyl groups (—CH₂OH) are placed at both terminals of a perfluoropolyether chain corresponding to R² (═R⁵═R⁵) in Formula (1). Then, a hydroxyl group of the hydroxymethyl group placed at one terminal of the fluorine compound is reacted with an epoxy compound having a group corresponding to R¹ (═R⁷) in Formula (1) (second reaction). As a result, an intermediate compound 2 having the group corresponding to R¹ (═R⁷) at one terminal of the perfluoropolyether chain corresponding to R² (═R⁶) is obtained.

The epoxy compound having the group corresponding to R¹ (═R⁷) may be reacted with the above-described fluorine compound after protecting the hydroxyl group with a suitable protecting group.

Epoxy compounds which are used in the second reaction when producing the fluorine-containing ether compound of the present embodiment can be synthesized, for example, by reacting alcohols having a structure corresponding to R¹ (or R⁷) of the fluorine-containing ether compound to be produced with a compound having any epoxy group selected from epichlorohydrin, epibromohydrin. 2-bromoethyloxirane, and allyl glycidyl ether. Such epoxy compounds may be synthesized through a method of oxidizing an unsaturated bond, or commercially available products may be purchased and used.

Thereafter, the hydroxyl group of the hydroxymethyl group placed at one terminal of the intermediate compound 2 is reacted with the epoxy groups placed at both terminals of the intermediate compound 1 (third reaction).

By performing the above-described steps, a compound in which three PFPE chains represented by R², R⁴, and R⁶ in Formula (1) have the same structure, R¹ is the same as R⁷, and R³ is the same as R⁶ can be produced. Here, the order of the first and second reactions may be reversed.

In addition, in a case where a compound in which R¹ and R⁷ are the same as each other and R¹ is different from R⁷ is produced as the fluorine-containing ether compound represented by Formula (1), the production method shown below can be used.

That is, in the above-described second reaction, an intermediate compound 2a having a group corresponding to R¹ and an intermediate compound 2b having a group corresponding to R¹ are synthesized. Thereafter, in the above-described third reaction, the compound can be produced through a method in which the intermediate compound 2a and the intermediate compound 2b are sequentially reacted with the epoxy group corresponding to R³ (═R″) placed at each terminal of the intermediate compound 1.

In addition, in a case where a compound in which R¹ and R⁷ are the same as each other and R³ is different from R⁵ is produced as the fluorine-containing ether compound represented by Formula (1), the production method shown below can be used.

That is, the halogen compound having the epoxy group corresponding to R³ and the halogen compound having the epoxy group corresponding to R⁵ are sequentially reacted with the hydroxyl group of the hydroxymethyl group placed at each terminal of the fluorine compound in the above-described first reaction. As a result, an intermediate compound 1a having the epoxy group corresponding to R³ at one end of the perfluoropolyether chain corresponding to R⁴ and the epoxy group corresponding to R⁵ at the other end is obtained. Thereafter, in the third reaction described above, the compound can be produced through a method in which the intermediate compound 1a is used instead of the intermediate compound 1.

Here, the function of a lubricating layer formed on a protective layer using a lubricant containing the fluorine-containing ether compound of the present embodiment will be described.

Examples of the cause of corrosion of a magnetic recording medium include ionic contamination substances present on the surface of the magnetic recording medium. Most of the ionic contamination substances adhere to the magnetic recording medium from outside during the production process of the magnetic recording medium. The ionic contamination substances may also be generated when environmental substances that have intruded into a hard disk drive (magnetic recording/reproducing device) adhere to the magnetic recording medium. Specifically, for example, water containing environmental substances such as ions may adhere to the surface of the magnetic recording medium when the magnetic recording medium and/or hard disk drive are held under high-temperature and high-humidity conditions. When water containing environmental substances such as ions passes through the lubricating layer formed on the surface of the magnetic recording medium, it condenses minute ionic components present under the lubricating layer to generate ionic contamination substances.

Since the fluorine-containing ether compound of the present embodiment is a compound represented by Formula (1), a lubricating layer containing this fluorine-containing ether compound exhibits excellent wear resistance and has a high corrosion suppressing effect that prevents contamination substances from intruding into the magnetic recording medium. This effect is based on synergistic effects <1> to <5> below obtained by incorporating the fluorine-containing ether compound of the present embodiment.

<1> The above-described lubricating layer adheres closely to a protective layer due to a suitable interaction generated between the protective layer and the lubricating layer due to one or more hydroxyl groups (—OH) included in each of R³ and R⁵ in the compound represented by Formula (1) and two or three hydroxyl groups each contained in R¹ and R⁷. For this reason, the lubricating layer having a high coating rate can be formed on the protective layer.

<2> In the fluorine-containing ether compound represented by Formula (1), three perfluoropolyether chains (R², R⁴, and R⁶ are each positioned between R¹ and R³, between R³ and R⁵, and between R⁵ and R⁷.

For this reason, the distance between a hydroxyl group in R³ and a hydroxyl group in R⁵, the distance between a hydroxyl group in R¹ and a hydroxyl group in R³, and the distance between a hydroxyl group in R¹ and a hydroxyl group in R⁷ are all appropriate. Accordingly, both the hydroxyl groups in R³ and R⁵ and the hydroxyl groups in R¹ and R⁷ are less likely to be inhibited from binding with active points on a protective layer due to the adjacent hydroxyl groups. Accordingly, both the hydroxyl groups in R¹ and R⁵ and the hydroxyl groups in R¹ and R⁷ are likely to participate in binding with the active points on the protective layer. In other words, all the hydroxyl groups in the above-described fluorine-containing ether compound are less likely to be hydroxyl groups that do not participate in binding with the active points on the protective layer. As a result, a lubricating layer containing the above-described fluorine-containing ether compound has a reduced number of hydroxyl groups that do not participate in binding with the active points on the protective layer and has excellent adhesion properties with respect to the protective layer.

In addition, since the distance between the hydroxyl group in R³ and the hydroxyl group in R⁵, the distance between the hydroxyl group in R¹ and the hydroxyl group in R³, and the distance between the hydroxyl group in R⁵ and the hydroxyl group in R⁷ are all appropriate, intramolecular interaction between the hydroxyl groups in R³, R⁵, R¹, and R⁷ is small and aggregation is less likely to occur. Accordingly, the fluorine-containing ether compound represented by Formula (1) is likely to wet and spread on the protective layer, and a lubricating layer having a uniform coating state and a high coating rate, and favorable adhesion properties, is formed on the protective layer.

Furthermore, both end portions of each perfluoropolyether chain (R², R⁴, and R⁶) adheres closely to the protective layer due to the hydroxyl groups in any of R³, R⁵, R¹, and R⁷. For this reason, the fluorine-containing ether compound applied onto the protective layer is less likely to be bulky. Accordingly, the fluorine-containing ether compound is likely to wet and spread on the protective layer, and a lubricating layer having a uniform coating state and a high coating rate, and favorable adhesion properties, is formed on the protective layer.

<3> The fluorine-containing ether compound represented by Formula (1) has three perfluoropolyether chains (R², R⁴, and R⁶) having the same structure. Accordingly, the fluorine-containing ether compound represented by Formula (1) is free from molecular strain due to the difference in mobility of PEPE chains occurring in a case where the PPPE chains have different structures, and is likely to wet and spread on the protective layer. Accordingly, a lubricating layer which has a uniform coating state, a high coating rate, and favorable adhesion properties is formed on the protective layer.

<4> The fluorine-containing ether compound represented by Formula (1) has three perfluoropolyether chains (R², R⁴, and R⁶). The perfluoropolyether chains contained in a lubricating layer cover the surface of the protective layer and impart water resistance to the lubricating layer due to their low surface energy.

The fluorine-containing ether compound represented by Formula (1) contained in the lubricating layer undergoes heat-induced molecular motion when held under high-temperature and high-humidity conditions. Water containing environmental substances such as ions is thought to enter through gaps between molecules in molecular motion in the lubricating layer.

Since the lubricating layer containing the fluorine-containing ether compound represented by Formula (1) has appropriate water resistance and hydrophobicity, it prevents water from entering the inside of the magnetic recording medium through the gaps between the molecules in molecular motion in the lubricating layer and improves corrosion resistance of the magnetic recording medium.

<5> R¹ and R⁷ in the fluorine-containing ether compound represented by Formula (1) are each independently a terminal group represented by Formula (2).

R¹ and R⁷ in Formula (1) each contains two or three hydroxyl groups, carbon atoms to which the hydroxyl group is bound are bound to each other via a linking group containing a carbon atom to which no hydroxyl group is bound, and the distance between the hydroxyl groups is appropriate. Accordingly, the lubricating layer containing the fluorine-containing ether compound represented by Formula (1) has appropriate hydrophobicity due to hydrophobicity of carbon atoms contained in the linking groups in R¹ and R⁷.

Furthermore, in the fluorine-containing ether compound represented by Formula (1), in a case where a linking group between a carbon atom to which a terminal hydroxyl group is bound and a carbon atom to which a hydroxyl group adjacent to the terminal hydroxyl group is bound does not contain an oxygen atom in each of R¹ and R⁷ (in a case where [E] in Formula (2) is a chain structure consisting of one to four methylene groups), intramolecular interaction between the hydroxyl groups contained in R¹ and R⁷ is small. For this reason, intramolecular aggregation is less likely to occur, the fluorine-containing ether compound is likely to wet and spread on the protective layer, and a lubricating layer having a uniform coating state and a high coating rate, and favorable adhesion properties, is formed on the protective layer.

In addition, even in a case where a linking group between a carbon atom to which a terminal hydroxyl group is bound and a carbon atom to which a hydroxyl group adjacent to the terminal hydroxyl group is bound contains an oxygen atom in each of R¹ and R⁷ (in a case where [E] in Formula (2) is a chain structure consisting of a combination of two to five methylene groups and one oxygen atom), the distance between the hydroxyl groups contained in R¹ and R⁷ is appropriate, and therefore, molecular mobility becomes appropriate. As a result, intramolecular aggregation is less likely to occur, and a lubricating layer which has a uniform coating state, a high coating rate, and favorable adhesion properties is formed on the protective layer.

### [Lubricant for Magnetic Recording Medium]

A lubricant for a magnetic recording medium of the present embodiment contains the fluorine-containing ether compound represented by Formula (1).

The lubricant of the present embodiment can be used after being mixed as necessary with a well-known material that is used as a material for lubricants within the scope not impairing the characteristics which are obtained due to the incorporation of the fluorine-containing ether compound represented by Formula (1).

Specific examples of well-known materials include FOMBLIN (registered trademark) ZDIAC, FOMBLIN ZDEAL, and FOMBLIN AM-2001 (all manufactured by Solvay Solexis), and Moresco A20H (manufactured by Moresco Corporation). The number average molecular weight of the well-known material that is used by being mixed with the lubricant of the present embodiment is preferably 1,000 to 10,000.

In a case where the lubricant of the present embodiment contains a material other than the fluorine-containing ether compound represented by Formula (1), the content of the fluorine-containing ether compound represented by Formula (1) in the lubricant of the present embodiment is preferably 50 masse or more and more preferably 70 mass % or more. The content of the fluorine-containing ether compound represented by Formula (1) may be 80 mass % or more or 90 mass % or more.

Since the lubricant of the present embodiment contains the fluorine-containing ether compound represented by Formula (1), a lubricating layer which exhibits an excellent wear resistance and is highly effective in suppressing corrosion of a magnetic recording medium can be formed. The lubricating layer consisting of the lubricant of the present embodiment exhibits an excellent wear resistance and is highly effective in suppressing corrosion of a magnetic recording medium, and therefore can be made thin.

### [Magnetic Recording Medium]

A magnetic recording medium of the present embodiment includes at least a magnetic layer, a protective layer, and a lubricating layer sequentially provided on a substrate.

In the magnetic recording medium of the present embodiment, one or more underlayers can be provided as necessary between the substrate and the magnetic layer. In addition, it is also possible to provide an adhesive layer and/or a soft magnetic layer between the underlayer and the substrate.

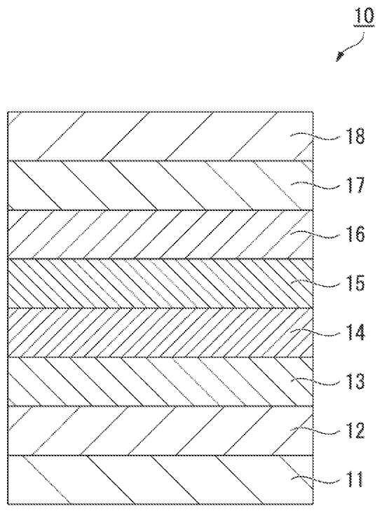

FIG. 1 is a schematic cross-sectional view showing one embodiment of a magnetic recording medium of the present invention.

A magnetic recording medium 10 of the present embodiment has a structure in which an adhesive layer 12, a soft magnetic layer 13, a first underlayer 14, a second underlayer 15, a magnetic layer 16, a protective layer 17, and a lubricating layer 18 are sequentially provided on a substrate 11.

### “Substrate”

As the substrate 11, for example, a non-magnetic substrate or the like in which a NiP or NiP alloy film is formed on a base made of metal or alloy material such as Al or an Al alloy can be used.

In addition, as the substrate 11, a non-magnetic substrate made of a non-metal material such as glass, ceramics, silicon, silicon carbide, carbon or a resin may be used, and a non-magnetic substrate in which a NIP or NiP alloy filet is formed on a base made of this non-metal material may also be used.

### “Adhesive Layer”

The adhesive layer 12 prevents the progress of corrosion of the substrate 11 which may occur in a case where the substrate 11 and the soft magnetic layer 13, which is provided on the adhesive layer 12, are arranged in contact with each other.

The material of the adhesive layer 12 can be appropriately selected from, for example, Cr, a Cr alloy, Ti, a Ti alloy, CrTi, NiAl, and an AlRu alloy. The adhesive layer 12 can be formed by, for example, a sputtering method.

### “Soft Magnetic Layer”

The soft magnetic layer 13 preferably has a structure in which a first soft magnetic filmi, an interlayer made of a Ru film, and a second soft magnetic film are sequentially laminated. That is, the soft magnetic layer 13 preferably has a structure in which the interlayer made of a Ru film is sandwiched between the two soft magnetic films, whereby the soft magnetic films on and under the interlayer are antiferromagnetically coupled (AFC).

Examples of the material of the first soft magnetic film and the second soft magnetic film include a CoZrTa alloy and a CoFe alloy.

Any of Zr, Ta and Nb is preferably added to the Cofe alloy that is used for the first soft magnetic film and the second soft magnetic film. This accelerates the amorphization of the first soft magnetic film and the second soft magnetic film, makes it possible to improve the orientation of the first underlayer (seed layer) and makes it possible to reduce the flying height of a magnetic head.

The soft magnetic layer 13 can be formed by, for example, a sputtering method.

### “First Underlayer”

The first underlayer 14 is a layer for controlling the orientations and crystal sizes of the second underlayer 15 and the magnetic layer 16 that are provided on the first underlayer 14.

Examples of the first underlayer 14 include a Cr layer, a Ta layer, a Ru layer, a CrMo alloy layer, a CoW alloy layer, a CrW alloy layer, a CrV alloy layer, and a Cr′Ti alloy layer.

The first underlayer 14 can be formed by, for example, a sputtering method.

### “Second Underlayer”

The second underlayer 15 is a layer that controls the orientation of the magnetic layer 16 to be favorable. The second underlayer 15 is preferably a Ra or Ra alloy layer.

The second underlayer 15 may be a single layer or may be composed of a plurality of layers. In a case where the second underlayer 15 is composed of a plurality of layers, all of the layers may be composed of the same material or at least one layer may be composed of a different material.

The second underlayer 15 can be formed by, for example, a sputtering method.

### “Magnetic Layer”

The magnetic layer 16 is made of a magnetic film in which the easy magnetization axis is directed in a perpendicular or parallel direction with respect to the substrate surface. The magnetic layer 16 is a layer containing Co and Pt and may be a layer further containing an oxide or Cr, B, Cu, Ta, Zr, or the like in order to improve SNR characteristics.

Examples of the oxide that is contained in the magnetic layer 16 include SiO₂, SiO, Cr₂O₃, CoO, Ta₂O₃, and TiO₂.

The magnetic layer 16 may be composed of a single layer or may be composed of a plurality of magnetic layers made of materials with different compositions.

For example, in a case where the magnetic layer 16 is composed of three layers of a first magnetic layer, a second magnetic layer, and a third magnetic layer sequentially laminated from below, the first magnetic layer is preferably a granular structure made of a material containing Co, Cr, and Pt and further containing an oxide. As the oxide that is contained in the first magnetic layer, for example, oxides of Cr, Si, Ta, Al, Ti, Mg, Co, or the like are preferably used. Among them, in particular, TiO₂, Cr₂O₃, SiO₂, and the like can be suitably used. In addition, the first magnetic layer is preferably made of a composite oxide to which two or more oxides have been added. Among them, in particular, Cr₂O₃—SiO₂, Cr₂O₃—TiO₂, SiO₂—TiO₂, and the like can be suitably used.

The first magnetic layer may contain, in addition to Co, Cr, Pt, and the oxide, one or more elements selected from the group consisting of B, Ta, Mo, Cu, Nd, W, Nb, Sm, Tb, Ru, and Re.

For the second magnetic layer, the same material as for the first magnetic layer can be used. The second magnetic layer preferably has a granular structure.

The third layer preferably has a non-granular structure made of a material containing Co, Cr, and Pt but containing no oxides. The third magnetic layer may contain, in addition to Co, Cr, and Pt, one or more elements selected from the group consisting of B, Ta, Mo, Co, Nd, W, Nh, Sm, Th, Ru, Re, and Mn.

In a case where the magnetic layer 16 is formed of a plurality of magnetic layers, a non-magnetic layer is preferably provided between the magnetic layers adjacent to each other. In a case where the magnetic layer 16 is made up of three layers of the first magnetic layer, the second magnetic layer and the third magnetic layer, it is preferable to provide a non-magnetic layer between the first magnetic layer and the second magnetic layer and a non-magnetic layer between the second magnetic layer and the third magnetic layer.

For the non-magnetic layer that is provided between the magnetic layers adjacent to each other in the magnetic layer 16, it is possible to suitably use, for example, Ru, a Ru alloy, a CoCr alloy, and a CoCrX1 alloy (XI represents one or, two or more elements selected from the group consisting of Pt, Ta, Zr, Re, Ru, Cu, Nb. Ni, Mn, Ge, Si, O, N, W, Mo, Ti, V, and B).

For the non-magnetic layer that is provided between the magnetic layers adjacent to each other in the magnetic layer 16, an alloy material containing an oxide, a metallic nitride or a metallic carbide is preferably used. Specifically, as the oxide, for example. SiO₂, Al₂O₃, Ta₂O₅, Cr₂O₃, MgO, Y₂O₃, and TiO₂ can be used. As the metallic nitride, for example, AlN, SiNa, TaN, and CrN can be used. As the metallic carbide, for example, TaC, BC, and SiC can be used.

The non-magnetic layer can be formed by, for example, a sputtering method.

The magnetic layer 16 is preferably a magnetic layer for perpendicular magnetic recording in which the easy magnetization axis is directed in a direction perpendicular to the substrate surface in order to realize a higher recording density. The magnetic layer 16 may be a magnetic layer for in-plane magnetic recording.

The magnetic layer 16 may be formed by any well-known conventional method such as a deposition method, an ion beam sputtering method, or a magnetron sputtering method. The magnetic layer 16 is normally formed by a sputtering method.

### “Protective Layer”

The protective layer 17 protects the magnetic layer 16. The protective layer 17 may be composed of a single layer or may be composed of a plurality of layers. As the material of the protective layer 17, carbon, nitrogen-containing carbon, silicon carbide, and the like can be exemplified.

As the protective layer 17, a carbon-based protective layer can be preferably used, and, in particular, an amorphous carbon protective layer is preferable. When the protective layer 17 is a carbon-based protective layer, an interaction with the hydroxyl group contained in the fluorine-containing ether compound in the lubricating layer 18 is further enhanced, which is preferable.

The adhesive force between the carbon-based protective layer and the lubricating layer 18 can be controlled by forming the carbon-based protective layer with hydrogenated carbon and/or nitrogenated carbon and adjusting the hydrogen content and/or the nitrogen content in the carbon-based protective layer. The hydrogen content in the carbon-based protective layer is preferably 3 to 20 atomic % when measured by the hydrogen forward scattering method (HFS). In addition, the nitrogen content in the carbon-based protective layer is preferably 4 to 15 atomic % when measured by X-ray photoelectron spectroscopy (XPS).

The hydrogen and/or nitrogen that are contained in the carbon-based protective layer do not need to be uniformly contained throughout the entire carbon-based protective layer. The carbon-based protective layer is suitably formed as, for example, a composition gradient layer in which nitrogen is contained in the lubricating layer 18 side of the protective layer 17 and hydrogen is contained in the magnetic layer 16 side of the protective layer 17. In this case, the adhesive force between the magnetic layer 16 and the carbon-based protective layer and the adhesive force between the lubricating layer 18 and the carbon-based protective layer further improve.

The film thickness of the protective layer 17 is preferably set to 1 am to 7 nm. When the film thickness of the protective layer 17 is 1 nm or more, performance as the protective layer 17 can be sufficiently obtained. The film thickness of the protective layer 17 is preferably 7 nm or less from the viewpoint of reducing the thickness of the protective layer 17.

As a method for forming the protective layer 17, it is possible to use a sputtering method in which a carbon-containing target material is used, a chemical vapor deposition (CVD) method in which a hydrocarbon raw material such as ethylene or toluene is used, an ion beam deposition (IBD) method, and the like.

In the case of forming a carbon-based protective layer as the protective layer 17, the carbon-based protective layer can be formed by, for example, a DC magnetron sputtering method. Particularly, in the case of forming a carbon-based protective layer as the protective layer 17, an amorphous carbon protective layer is preferably formed by a plasma CVD method. The amorphous carbon protective layer formed by the plasma CVD method has a uniform surface with small roughness,

### “Lubricating Layer”

The lubricating layer 18 prevents contamination of the magnetic recording medium 10. In addition, the lubricating layer 18 reduces frictional force of a magnetic head of a magnetic recording/reproducing device, which slides on the magnetic recording medium 10, thereby improving the durability of the magnetic recording medium 10. The lubricating layer 18 is formed in contact with the protective layer 17 as shown in FIG. 1. The lubricating layer 18 contains the above-described fluorine-containing ether compound.

In a case where the protective layer 17, which is placed below the lubricating layer 18, is a carbon-based protective layer, particularly, the lubricating layer 18 is bound to the protective layer 17 with a high binding force. As a result, the magnetic recording medium 10 in which the surface of the protective layer 17 is coated with the lubricating layer 18 at a high coating rate in spite of a thin thickness is likely to be obtained, and contamination on the surface of the magnetic recording medium 10 can be effectively prevented.

The average film thickness of the lubricating layer 18 is preferably 0.5 nm (5 Å) to 2.0 nm (20 Å) and more preferably 0.5 nm (5 Å) to 1.0 nm (10 Å). When the average film thickness of the lubricating layer 18 is 0.5 nm or more, the lubricating layer 18 does not have an island shape or a mesh shape and is formed in a uniform film thickness. For this reason, the surface of the protective layer 17 can be coated with the lubricating layer 18 at a high coating rate. In addition, when the average film thickness of the lubricating layer 18 is set to 2.0 nm or less, it is possible to sufficiently reduce the thickness of the lubricating layer 18 and to sufficiently decrease the flying height of a magnetic head.

In a case where the surface of the protective layer 17 is not sufficiently coated with the lubricating layer 18 at a high coating rate, an environmental substance adsorbed to the surface of the magnetic recording medium 10 passes through voids in the lubricating layer 18 and intrudes below the lubricating layer 18. The environmental substance that has intruded below the lubricating layer 18 is adsorbed and bound to the protective layer 17 and generates a contamination substance. At the time of reproducing magnetic records, the generated contamination substance (aggregated component) adheres (transfers) to a magnetic head as a smear to break the magnetic head or degrade the magnetic recording/reproducing characteristics of magnetic recording/reproducing devices.

Examples of the environmental substance that generates the contamination substance include siloxane compounds (cyclic siloxane and linear siloxane), ionic impurities, hydrocarbons having a relatively high molecular weight such as octacosane, and plasticizers such as dioctyl phthalate. Examples of metal ions contained in the ionic impurities include a sodium ion and a potassium ion. Examples of inorganic ions contained in the ionic impurities include a chlorine ion, a bromine ion, a nitrate ion, a sulfate ion, and an ammonium ion. Examples of organic ions contained in the ionic impurities include an oxalate ion and a formate ion.

### “Method for Forming Lubricating Layer”

Examples of methods for forming the lubricating layer 18 include a method in which a magnetic recording medium that is not yet fully manufactured and thus includes the individual layers up to the protective layer 17 formed on the substrate 11 is prepared and a solution for forming a lubricating layer is applied onto the protective layer 17 and dried.

The solution for forming a lubricating layer can be obtained by dispersing and dissolving the above-described lubricant for a magnetic recording medium of the embodiment in a solvent as necessary and adjusting the viscosity and concentration to be suitable for application methods.

Examples of solvents used for the solution for forming a lubricating layer include fluorine-based solvents such as VERTREL (registered trademark) XF (trade name, manufactured by Dupont-Mitsui Fluorochemicals Co., Ltd.).

A method for applying the solution for forming a lubricating layer is not particularly limited, and examples thereof include a spin coating method, a spraying method, a paper coating method, and a dipping method.

In a case of using a dipping method, it is possible to use, for example, a method shown below. First, the substrate 11 on which the individual layers up to the protective layer 17 have been formed is immersed in the solution for forming a lubricating layer that has been placed in an immersion vessel of a dip coater. Next, the substrate 11 is lifted from the immersion vessel at a predetermined speed. As a result, the solution for forming a lubricating layer is applied to the surface of the protective layer 17 on the substrate 11.

The use of the dipping method makes it possible to uniformly apply the solution for forming a lubricating layer to the surface of the protective layer 17 and makes it possible to form the lubricating layer 18 on the protective layer 17 in a uniform film thickness.

In the present embodiment, a burnishing (precision polishing) step is preferably performed after the lubricating layer 18 is formed on the surface of the substrate 11. By performing the burnishing step, projection defects and particles present on the surface of the substrate 11 on which the lubricating layer 18 has been formed can be removed, and the magnetic recording medium 10 with a smooth surface can be obtained. If the surface of the magnetic recording medium 10 is smooth, the spacing loss between the magnetic recording medium 10 and a magnetic head can be reduced and the signal characteristics can improve.

As the burnishing step, for example, a step of scanning burnishing tape on the surface of the substrate 11 on which the lubricating layer 18 has been formed can be performed. As the burnishing tape, one made of a resin film holding abrasive grains can be used. The grain size of the abrasive grains can be set to, for example, #6000 to #20000.

In the present embodiment, a heat treatment is preferably carried out on the substrate 11 on which the lubricating layer 18 has been formed. The heat treatment improves the adhesion properties between the lubricating layer 18 and the protective layer 17 and improves the adhesive force between the lubricating layer 18 and the protective layer 17.

The heat treatment temperature is preferably set to 100° ° C., to 180° ° C. When the heat treatment temperature is 100° C. or higher, an effect on improvement in the adhesion properties between the lubricating layer 18 and the protective layer 17 can be sufficiently obtained. In addition, when the heat treatment temperature is set to 180° ° C. or lower, it is possible to prevent thermal decomposition of the lubricating layer 18. The heat treatment time is preferably set to 10 to 120 minutes.

The magnetic recording medium 10 of the present embodiment includes at least the magnetic layer 16, the protective layer 17, and the lubricating layer 18 sequentially provided on the substrate 11. In the magnetic recording medium 10 of the present embodiment, the lubricating layer 18 containing the above-described fluorine-containing ether compound is formed in contact with the protective layer 17. This lubricating layer 18 is highly effective in suppressing corrosion of the magnetic recording medium 10. For this reason, the magnetic recording medium 10 of the present embodiment has less contamination substances present on the surface, an excellent corrosion resistance, and favorable reliability and durability. In addition, since the magnetic recording medium of the present embodiment has the lubricating layer 18 highly effective in suppressing corrosion, the thickness of the protective layer 17 and/or the lubricating layer 18 can be reduced. In addition, in the lubricating layer 18 in the magnetic recording medium 10 of the present embodiment, foreign matter (smears) are less likely to be generated, and pickup can be suppressed.

### EXAMPLES

Hereinafter, the present invention will be described more specifically with reference to examples and comparative examples. The present invention is not limited to the following examples.

### Example 1

A compound represented by Formula (A) above was produced through a method shown below.

### (First Reaction)

9.4 g (20 mmol) of a compound represented by HOCH₂CF₂O(CF₂CF₂O)ₚCF₂CH₂OH (p indicating the average degree of polymerization in the formula is 2.5) (number average molecular weight: 468, molecular weight distribution: 1.1), 1.76 g (44 mmol) of 60% sodium hydride, and 15.6 mL of N,N-dimethylformamide were added to a 200 mL eggplant flask in a nitrogen gas atmosphere and stirred until the mixture became uniform at room temperature. 3.45 mL of epibromohydrin (42 mmol) was further added to this uniform solution and reacted by being stirred at 40° C. for 2 hours.

A reaction product obtained after the reaction was cooled to 25° C. 80 mL of water was added thereto to stop the reaction, the mixture was moved to a separatory funnel, and extraction was performed twice with 150 mL of ethyl acetate. An organic layer thereof was washed with saturated saline and dehydrated with anhydrous sodium sulfate. After the drying agent was filtered, the filtrate was concentrated, and the residue was purified by silica gel column chromatography, thereby obtaining 3.5 g (molecular weight: 580, 6.0 mmol) of a compound represented by Formula (10) below as an intermediate compound 1.

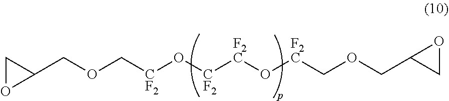

(In Formula (10), p indicating the average degree of polymerization is 2.5.)

### (Second Reaction)

14.0 g of the compound represented by HOCH₂CF₂O(CF₂CF₂O)ₚCF₂CH₂OH (p indicating the average degree of polymerization in the formula is 2.5) (number average molecular weight: 468, molecular weight distribution: 1.1), 3.4 g (molecular weight: 186.25, 18 mmol) of a compound represented by Formula (11) below, and 28 mL of t-butanol were added to a 200 ml eggplant flask in a nitrogen gas atmosphere and stirred until the mixture became uniform at room temperature. 1.0 g (molecular weight: 112.2, 9 mmol) of potassium tert-butoxide was further added to this uniform solution and reacted by being stirred at 70° C. for 16 hours.

The compound represented by Formula (11) was synthesized by introducing a tetrahydropyranyl (THP) group into a primary hydroxyl group in 4-penten-1-ol and oxidizing the double bond of the obtained compound.

A reaction product obtained after the reaction was cooled to 25° C., moved to a separatory funnel containing 100 ml of water, and extracted three times with 100 mL of ethyl acetate. An organic layer thereof was washed with water and dehydrated with anhydrous sodium sulfate. After the drying agent was filtered, the filtrate was concentrated, and the residue was purified by silica gel column chromatography, thereby obtaining 7.9 g (molecular weight: 654, 12.0 mmol) of a compound represented by Formula (12) below as an intermediate compound 2.

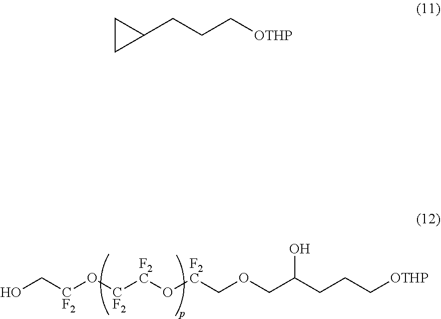

(In Formula (11), THP represents a tetrahydropyranyl group.)

(In Formula (12), p indicating the average degree of polymerization is 2.5.)

### (Third Reaction)

6.5 g of the intermediate compound 2 represented by Formula (12) (p indicating the average degree of polymerization in the formula is 2.5), 0.34 g of potassium tert-butoxide, and 9.4 mL of t-butanol were added to a 200 mL eggplant flask in a nitrogen gas atmosphere and stirred until the mixture became uniform at room temperature. 1.7 g of the intermediate compound 1 represented by Formula (10) (p indicating the average degree of polymerization in the formula is 2.5) was further added to this uniform liquid and reacted by being stirred at 70° ° C. for 16 hours.

A reaction product obtained after the reaction was cooled to 25° C., moved to a separatory funnel containing 100 ml of water, and extracted three times with 100 ml of ethyl acetate. An organic layer thereof was washed with water and dehydrated with anhydrous sodium sulfate. After the drying agent was filtered, the filtrate was concentrated, and the residue was purified by silica gel column chromatography, thereby obtaining 3.1 g (molecular weight: 1720, 1.8 mmol) of the compound represented by Formula (A) above (Fpa₁ and Fpa₂ in Formula (A) are represented by Formula (AF), pa indicating the average degree of polymerization in Fpa₁ is 2.5, and pa indicating the average degree of polymerization in Fpa₂ is 2.5).

¹H-NMR and ¹⁹F-NMR measurements of the obtained compound (A) were carried out, and the structure was identified from the following results.

¹H-NMR (CD₃COCD₃): δ [ppm]=1.38 to 1.75 (8H), 3.37 to 4.31 (38H)

¹⁹F-NMR (CD₃COCD₃): δ [ppm]=−78.6 (6F), −81.3 (6F), −90.0 to −88.5 (30F)

### Example 2

The same operation as in Example 1 was carried out except that 13.9 g of a compound represented by HOCH₂CF₂CF₂O(CF₂CF₂CF₂O)_{q}CF₂CF₂CH₂OH (q indicating the average degree of polymerization in the formula is 2.5) (number average molecular weight: 693, molecular weight distribution: 1.1) was used instead of the compound represented by HOCH₂CF₂O(CF₂CF₂O)ₚCF₂CH₂OH (p indicating the average degree of polymerization in the formula was 2.5) in the first reaction, and that 20.8 g of a compound represented by HOCH₂CF₂CF₂O(CF₂CF₂CF₂O)_{q}CF₂CF₂CH₂OH (q indicating the average degree of polymerization in the formula is 2.5) (number average molecular weight: 693, molecular weight distribution: 1.1) was used instead of the compound represented by HOCH₂CF₂O(CF₂CF₂O)ₚCF₂CH₂OH (p indicating the average degree of polymerization in the formula was 2.5) and 3.61 g of a compound represented by Formula (13) below was used instead of the compound represented by Formula (11) in the second reaction, thereby obtaining 4.4 g (molecular weight: 2424, 1.8 mmol) of the compound represented by Formula (B) above (Fdb₁ and Fdb₂ in Formula (B) are represented by Formula (BF), qb indicating the average degree of polymerization in Fdb₁ is 2.5, and qb indicating the average degree of polymerization in Fdb₂ is 2.5).

The compound represented by Formula (13) was synthesized by introducing a tetrahydropyranyl (THP) group into a primary hydroxyl group in 5-hexen-1-ol and oxidizing the double bond of the obtained compound.

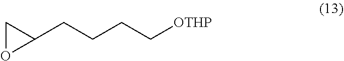

(In Formula (13), THP represents a tetrahydropyranyl group.)

¹H-NMR and ¹⁹F-NMR measurements of the obtained compound (B) were carried out, and the structure was identified from the following results.

¹H-NMR (CD₃COCD₃): δ [ppm]=1.38 to 1.81 (12H), 3.36 to 4.35 (38H)

¹⁹F-NMR (acetone-D₆): δ [ppm]=−84.0 to −83.0 (30F), −86.4 (12F), −124.3 (12F), −130.0 to −129.0 (15F)

### Example 3

The same operation as in Example 1 was carried out except that 12.7 g of a compound represented by HOCH₂CF₂O(CF₂CF₂O)ₘ(CF₂O)ₙCF₂CH₂OH (m and n indicating the average degree of polymerization in the formula are 2.5) (number average molecular weight: 633, molecular weight distribution: 1.1) was used instead of the compound represented by HOCH₂CF₂O(CF₂CF₂O)ₚCF₂CH₂OH (p indicating the average degree of polymerization in the formula is 2.5) in the first reaction, and that 19.0 g of the compound represented by HOCH₂CF₂O(CF₂CF₂O)ₘ(CF₂O)ₙCF₂CH₂OH (m and n indicating the average degree of polymerization in the formula are 2.5) (number average molecular weight: 633, molecular weight distribution: 1.1) was used instead of the compound represented by HOCH₂CF₂O(CF₂CF₂O)ₚCF₂CH₂OH (p indicating the average degree of polymerization in the formula is 2.5) and 3.89 g of a compound represented by Formula (14) below was used instead of the compound represented by Formula (11) in the second reaction, thereby obtaining 4.1 g (molecular weight: 2276, 1.8 mmol) of the compound represented by Formula (C) above (Ffc₁ and Ffc₂ in Formula (C) are represented by Formula (CF), mc and ne indicating the average degree of polymerization in Ffc₁ are 2.5, and me and ne indicating the average degree of polymerization in Ffc₂ are 2.5).

The compound represented by Formula (14) was synthesized by protecting one hydroxyl group of 1,3-propanediol with a tetrahydropyranyl (THP) group and reacting epibromohydrin with the other hydroxyl group.

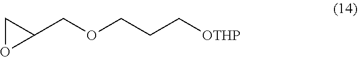

(In Formula (14). THP represents a tetrahydropyranyl group.)

¹H-NMR and ¹⁹F-NMR measurements of the obtained compound (C) were carried out, and the structure was identified from the following results.

¹H-NMR (CD₃COCD₃): δ [ppm]=1.34 to 1.67 (4H), 3.39 to 4.34 (46H)

¹⁹F-NMR (CD₃COCD₃): δ [ppm]=55.6 to −50.6 (15F), −77.7 (6F), −80.3 (6F), −91.0 to −88.5 (30F)

### Example 4

The same operation as in Example 1 was carried out except that 4.15 g of a compound represented by Formula (15) below was used instead of the compound represented by Formula (11) in the second reaction, thereby obtaining 3.3 g (molecular weight: 1809, 1.8 mmol) of the compound represented by Formula (D) above (Fpd₁ and Fpd₂ in Formula (D) are represented by Formula (DF), pd indicating the average degree of polymerization in Fpd₁ is 2.5, and pd indicating the average degree of polymerization in Fpd₂ is 2.5).

The compound represented by Formula (15) was synthesized by protecting one hydroxyl group of 1,4-butanediol with a tetrahydropyranyl (THP) group and reacting epibromohydrin with the other hydroxyl group.

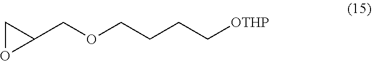

(In Formula (15), THP represents a tetrahydropyranyl group.)

¹H-NMR and ¹⁹F-NMR measurements of the obtained compound (D) were carried out, and the structure was identified from the following results.

1H-NMR (CD₃COCD₃): δ [ppm]=1.34 to 1.65 (8H), 3.42 to 4.35 (46H)

¹⁹F-NMR (CD₃COCD₃): δ [ppm]=−78.6 (6F), −81.3 (6F), −90.0 to −88.5 (30F)

### Example 5

The same operation as in Example 2 was carried out except that 5.77 g of a compound represented by Formula (16) below was used instead of the compound represented by Formula (13) in the second reaction, thereby obtaining 4.6 g (molecular weight: 2576, 1.8 mmol) of the compound represented by Formula (B) above (Fde₁ and Fde₂ in Formula (E) are represented by Formula (EF), qe indicating the average degree of polymerization in Fde₁ is 2.5, and qe indicating the average degree of polymerization in Fde₂ is 2.5).

The compound represented by Formula (16) was synthesized by the following method.

A tert-butyldimethylsilyl (TBS) group was introduced as a protective group into a primary hydroxyl group in 3-allyloxy-1,2-propanediol, and a methoxymethyl (MOM) group was introduced as a protective group into a secondary hydroxyl group in the obtained compound. After that, the TBS group was removed from the compound, and 2-bromoethoxytetrahydropyran was reacted with the generated primary hydroxyl group. The double bond of the obtained compound was oxidized. The compound represented by Formula (16) was obtained through the above-described steps.

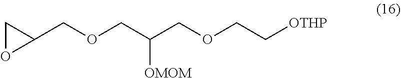

(In Formula (16), THP represents a tetrahydropyranyl group, and MOM represents a methoxymethyl group.)

¹H-NMR and ¹⁹F-NMR measurements of the obtained compound (E) were carried out, and the structure was identified from the following results.

¹H-NMR (CD₃COCD₃): δ [ppm]=3.31 to 4.41 (58H)

¹⁹F-NMR (acetone-D₆): δ [ppm]=−84.0 to −83.0 (30F), −86.4 (12F), −124.3 (12F), −130.0 to −129.0 (15F)

### Example 6

The same operation as in Example 2 was carried out except that 6.02 g of a compound represented by Formula (17) below was used instead of the compound represented by Formula (13) in the second reaction, thereby obtaining 4.7 g (molecular weight: 2604, 1.8 mmol) of the compound represented by Formula (F) above (Fdf₁ and Fdf₂ in Formula (F) are represented by Formula (FF), qf indicating the average degree of polymerization in Fdf₁ is 2.5, and qf indicating the average degree of polymerization in Fdf₂ is 2.5).

The compound represented by Formula (17) was synthesized by the following method.

A TBS group was introduced into the primary hydroxyl group in 3-allyloxy-1,2-propanediol, and a MOM group was introduced into a secondary hydroxyl group in the obtained compound. The TBS group in the obtained compound was removed, and 2-(chloropropoxy)tetrahydro-2H-pyran was reacted with the generated primary hydroxyl group. The double bond of the obtained compound was oxidized. The compound represented by Formula (17) was obtained through the above-described steps.

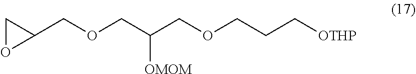

(In Formula (17), THP represents a tetrahydropyranyl group, and MOM represents a methoxymethyl group.)

¹H-NMR and ¹⁹F-NMR measurements of the obtained compound (F) were carried out, and the structure was identified from the following results.

¹H-NMR (CD₃COCD₃): δ [ppm]=1.34 to 1.59 (4H), 3.37 to 4.38 (58H)

¹⁹F-NMR (acetone-D₆): δ [ppm]=−84.0 to −83.0 (30F), −86.4 (12F), −124.3 (12F), −130.0 to −129.0 (15F)

### Example 7

The same operation as in Example 3 was carried out except that 3.89 g of a compound represented by Formula (18) below was used instead of the compound represented by Formula (14) in the second reaction, thereby obtaining 4.1 g (molecular weight: 2276, 1.8 mmol) of the compound represented by Formula (G) above (Ffg₁ and Ffg₂ in Formula (G) are represented by Formula (GF), mg and ng indicating the average degree of polymerization in Ffg₁ are 2.5, and mg and ng indicating the average degree of polymerization in Ffg₂ are 2.5).

The compound represented by Formula (18) was synthesized by oxidizing the double bond of a compound obtained by reacting 3-buten-1-ol with 2-bromoethoxytetrahydropyran.

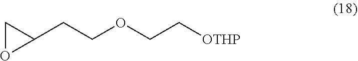

(In Formula (18), THP represents a tetrahydropyranyl group.)

¹H-NMR and ¹⁹F-NMR measurements of the obtained compound (G) were carried out, and the structure was identified from the following results.

¹H-NMR (CD₃COCD₃): δ [ppm]=1.34 to 1.67 (4H), 3.38 to 4.33 (46H)

¹⁹F-NMR (CD₃COCD₃): δ [ppm]=−55.6 to −50.6 (15F), −77.7 (6F), −80.3 (6F), 91.0 to −88.5 (30F)

### Example 8

The same operation as in Example 3 was carried out except that 6.02 g of a compound represented by Formula (19) below was used instead of the compound represented by Formula (14) in the second reaction, thereby obtaining 4.4 g (molecular weight: 2424, 1.8 mmol) of the compound represented by Formula (H) above (Ffh₁ and Ffh₂ in Formula (H) are represented by Formula (HF), mh and nh indicating the average degree of polymerization in Ffh₁ are 2.5, and mh and nh indicating the average degree of polymerization in Ffh₂ are 2.5).

The compound represented by Formula (19) was synthesized by the following method.

Ethylene glycol monoallyl ether was protected using dihydropyran and oxidized to obtain a first compound. The obtained first compound was reacted with a hydroxyl group of 3-buten-1-ol to obtain a second compound. A secondary hydroxyl group of the obtained second compound was protected with a MOM group, and then the double bond of the second compound was oxidized. The compound represented by Formula (19) was obtained through the above-described steps.

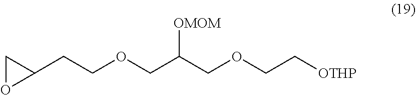

(In Formula (19), THP represents a tetrahydropyranyl group, and MOM represents a methoxymethyl group.)

¹H-NMR and ¹⁹F-NMR measurements of the obtained compound (H) were carried out, and the structure was identified from the following results.

¹H-NMR (CD₃COCD₃): δ [ppm]=3.41 to 4.36 (62H)

¹⁹F-NMR (CD₃COCD₃): δ [ppm]=−55.6 to −50.6 (15F), −77.7 (6F), 80.3 (6F), −91.0 to −88.5 (30F)

### Example 9

The same operation as in Example 1 was carried out except that 4.51 g of a compound represented by Formula (20) below was used instead of the compound represented by Formula (11) in the second reaction, thereby obtaining 3.3 g (molecular weight: 1841, 1.8 mmol) of the compound represented by Formula (D) above (Fpi₁ and Fpi₂ in Formula (1) are represented by Formula (1F), pi indicating the average degree of polymerization in Fpi₁ is 2.5, and pi indicating the average degree of polymerization in Fpi₂ is 2.5).

The compound represented by Formula (20) was synthesized by the following method.

1,2,4-Butanetriol was reacted with benzaldehyde dimethylacetal to synthesize a compound in which hydroxyl groups bound to 2- and 4-position carbons of 1,2,4-butanetriol were protected. This compound was reacted with epibromohydrin to synthesize the compound represented by Formula (20).

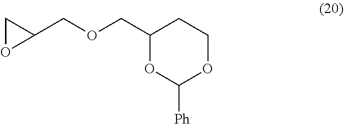 [SMILES: c1ccc(C2OCCC(COCC3CO3)O2)cc1]

(In Formula (20), Ph represents a phenyl group.)

¹H-NMR and ¹⁹F-NMR measurements of the obtained compound (I) were carried out, and the structure was identified from the following results.

¹H-NMR (CD₃COCD₃): δ [ppm]=1.34 to 1.58 (4H), 3.38 to 4.34 (60H)

¹⁹F-NMR (CD₃COCD₃): δ [ppm]=−78.6 (6F), −81.3 (6F), −90.0 to −88.5 (30F)

### Example 10

The same operation as in Example 1 was carried out except that 5.48 g of a compound represented by Formula (21) below was used instead of the compound represented by Formula (11) in the second reaction, thereby obtaining 3.4 g (molecular weight: 1869, 1.8 mmol) of the compound represented by Formula (J) above (Fpj₁ and Fpi₂ in Formula (J) are represented by Formula (JF), pj indicating the average degree of polymerization in Fpj₁ is 2.5, and pj indicating the average degree of polymerization in Fpj₂ is 2.5).

The compound represented by Formula (21) was synthesized by the following method.

A secondary hydroxyl group of a compound obtained by reacting the compound represented by Formula (11) with allyl alcohol was protected with a MOM group. The double bond of the obtained compound was oxidized to obtain the compound represented by Formula (21).

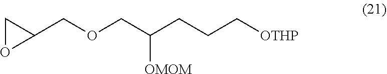

(In Formula (21). THP represents a tetrahydropyranyl group, and MOM represents a methoxymethyl group.)

¹H-NMR and ¹⁹F-NMR measurements of the obtained compound (J) were carried out, and the structure was identified from the following results.

¹H-NMR (CD₃COCD₃): δ [ppi]=1.34 to 1.66 (8H), 3.39 to 4.35 (60H)

¹⁹F-NMR (CD₃COCD₃): δ [ppm]=−78.6 (6F), −81.3 (6F), −90.0 to −88.5 (30F)

### Example 11

The same operation as in Example 2 was carried out except that 6.99 g of a compound represented by Formula (22) below was used instead of the compound represented by Formula (13) in the second reaction, thereby obtaining 4.7 g (molecular weight: 2632, 1.8 mmol) of the compound represented by Formula (K) above (Fdk₁ and Fdk₂ in Formula (K) are represented by Formula (KF), qk indicating the average degree of polymerization in Fdk₁ is 2.5, and qk indicating the average degree of polymerization in Fdk₂ is 2.5).

The compound represented by Formula (22) was synthesized by the following method.

Ethylene glycol monoallyl ether was protected using dihydropyran and oxidized to obtain a first compound. The obtained first compound was reacted with a hydroxyl group of 4-penten-1-ol to obtain a second compound. A secondary hydroxyl group of the obtained second compound was protected with a THP group, and then the double bond of the second compound was oxidized. The compound represented by Formula (22) was obtained through the above-described steps.

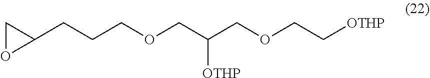

(In Formula (22), THP represents a tetrahydropyranyl group.)

¹H-NMR and ¹⁹F-NMR measurements of the obtained compound (K) were carried out, and the structure was identified from the following results.

¹H-NMR (CD₃COCD₃): δ [ppm]=1.34 to 1.63 (8H), 3.39 to 4.35 (38H)

¹⁹F-NMR (acetone-D₆): δ [ppm]=−84.0 to −83.0 (30F), −86.4 (12F), −124.3 (12F), −130.0 to ˜ 129.0 (15F)

### Example 12

The same operation as in Example 2 was carried out except that 4.76 g of a compound represented by Formula (23) below was used instead of the compound represented by Formula (13) in the second reaction, thereby obtaining 4.6 g (molecular weight: 2544, 1.8 mmol) of the compound represented by Formula (L) above (Fdl₁ and Fdl₂ in Formula (L) are represented by Formula (1F), ql indicating the average degree of polymerization in Fdl₁ is 2.5, and ql indicating the average degree of polymerization in Fdl₂ is 2.5).

The compound represented by Formula (23) was synthesized by the following method, 1,2,4-Butanetriol was reacted with benzaldehyde dimethylacetal to synthesize a compound in which hydroxyl groups bound to 2- and 4-position carbons of 1,2,4-butanetriol were protected. This compound was reacted with 2-bromoethyloxirane to synthesize the compound represented by Formula (23).

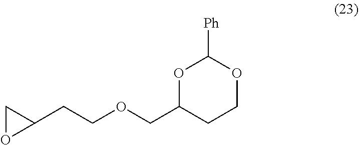 [SMILES: c1ccc(C2OCCC(COCCC3CO3)O2)cc1]

(In Formula (23), Ph represents a phenyl group.)

¹H-NMR and ¹⁹F-NMR measurements of the obtained compound (L) were carried out, and the structure was identified from the following results.

¹H-NMR (CD₃COCD₃): δ [ppm]=1.35 to 1.62 (8H), 3.41 to 4.35 (60H)

¹⁹F-NMR (acetone-D₆): δ [ppm]=−84.0 to −83.0 (30F), −86.4 (12F), −124.3 (12F), −130.0 to −129.0 (15F)

### Example 13

The same operation as in Example 2 was carried out except that 3.10 g of a compound represented by Formula (24) below was used instead of the compound represented by Formula (13) in the second reaction, thereby obtaining 4.3 g (molecular weight: 2368, 1.8 mmol) of the compound represented by Formula (M) above (Fdn₁ and Fdn₂ in Formula (M) are represented by Formula (MF), qm indicating the average degree of polymerization in Fdm₁ is 2.5, and qm indicating the average degree of polymerization in Fdm₂ is 2.5).

The compound represented by Formula (24) was synthesized by introducing a THP group into a primary hydroxyl group in 3-buten-1-ol and oxidizing the double bond of the obtained compound.

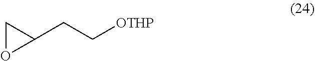

(In Formula (24), THP represents a tetrahydropyranyl group.)

¹H-NMR and ¹⁹F-NMR measurements of the obtained compound (M) were carried out, and the structure was identified from the following results.

¹H-NMR (CD₃COCD₃): δ [ppm]=1.34 to 1.57 (4H), 3.41 to 4.37 (38H)

¹⁹F-NMR (acetone-D₆): δ [ppm]=>84.0 to −83.0 (30F), −86.4 (12F), −124.3 (12F), −130.0 to ˜129.0 (15F)

### Comparative Example 1

A compound represented by Formula (N) below was synthesized by the method described in Patent Document 1.

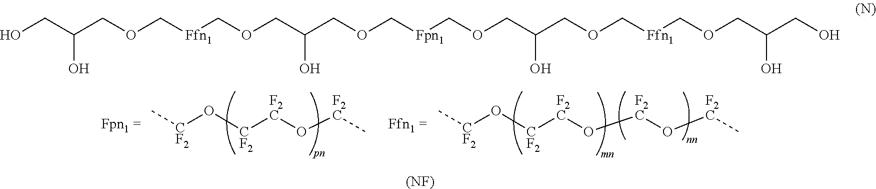

(Fpn₁ and Ffn₁ in Formula (N) are represented by Formula (NE), pn indicating the average degree of polymerization in Fpn₁ is 2.5, and mn and an indicating average degrees of polymerization in Ffn₁ each independently represent 2.5.)

¹H-NMR and ¹⁹F-NMR measurement of the obtained compound (N) were carried out, and the structure was identified from the following results.

¹H-NMR (CD₃COCD₃): δ [ppm]=3.42 to 4.28 (38H)

¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Comparative Example 2

A compound represented by Formula (O) below was synthesized by the method described in Patent Document 1.

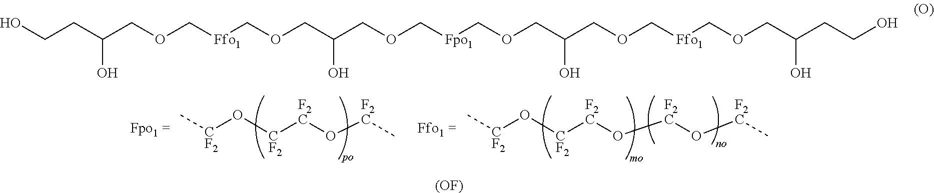

(Fpo₁ and Ffo₁ in Formula (O) are represented by Formula (0F), po indicating the average degree of polymerization in Fpo₁ is 2.5, and mo and no indicating average degrees of polymerization in Ffo₁ each independently represent 2.5.)

¹H-NMR and ¹⁹F-NMR measurement of the obtained compound (O) were carried out, and the structure was identified from the following results.

¹H-NMR (CD₃COCD₃): δ [ppm]=1.54 to 1.76 (4H), 3.42 to 4.28 (38H)

¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Comparative Example 3

A compound represented by Formula (P) below was synthesized by the method described in Parent Document 1.

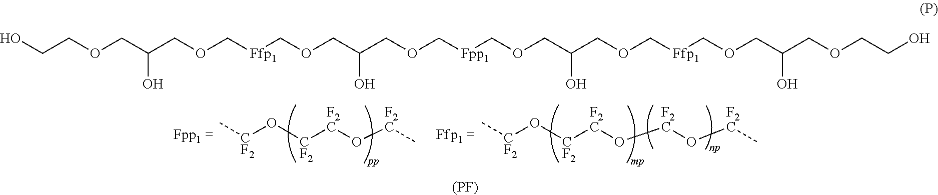

(Fpp₁ and Ffp₁ in Formula (P) are represented by Formula (PF), pp indicating the average degree of polymerization in Fpp₁ is 2.5, and mp and np indicating average degrees of polymerization in Ffp₁ each independently represent 2.5.)

¹H-NMR and ¹⁹F-NMR measurements of the obtained compound (P) were carried out, and the structure was identified from the following results.

¹H-NMR (CD₃COCD₃): δ [ppm]=3.46 to 4.18 (46H)

¹⁹F-NMR (CD₃COCD₃): δ [ppm]=−55.6 to −50.6 (10F), −77.7 (4F), −78.6 (2F), −80.3 (4F), −81.3 (2F, −90.0 to −88.5 (30F)

### Comparative Example 41

A compound represented by Formula (Q) below was synthesized by the method described in Patent Document 1,

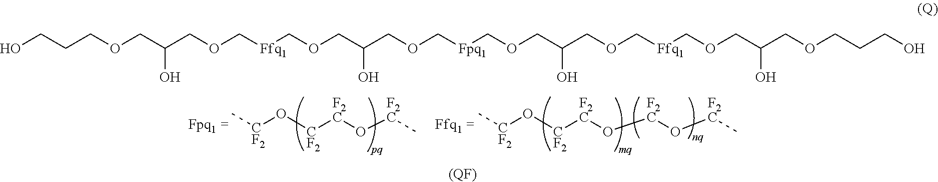

(Fpq₁ and Ffq₁ in Formula (Q) are represented by Formula (QF), pq indicating the average degree of polymerization in Fpq₁ is 2.5, and mq and nq indicating average degrees of polymerization in Ffq₁ each independently represent 2.5.)

¹H-NMR and ¹⁹F-NMR measurements of the obtained compound (Q) were carried out, and the structure was identified from the following results.

¹H-NMR (CD₃COCD₃): δ [ppm]=1.34 to 1.67 (4H), 3.39 to 4.34 (46H)

¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Comparative Example 5

A compound represented by Formula (R) below was synthesized by the method described in Patent Document 2.

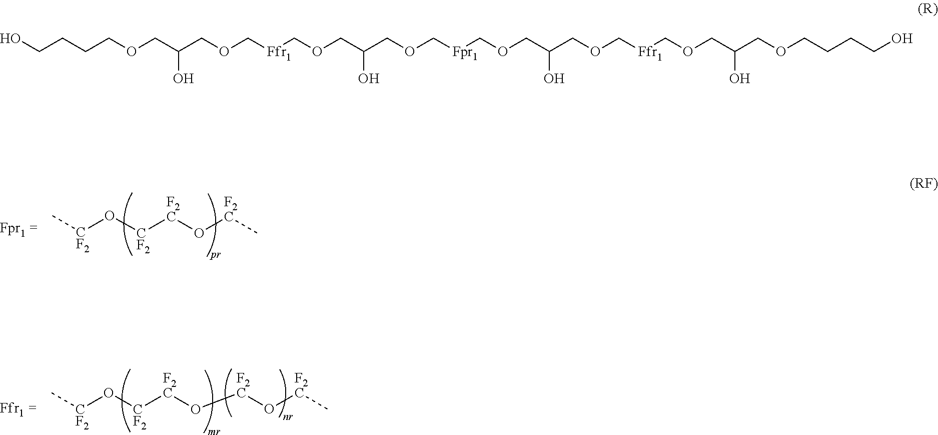

(Fpr₁ and Ffr₁ in Formula (R) are represented by Formula (RF), pr indicating the average degree of polymerization in Fpr₁ is 2.5, and mr and nr indicating average degrees of polymerization in Ffr₁ each independently represent 2.5.)

¹H-NMR and ¹⁹F-NMR measurements of the obtained compound (R) were carried out, and the structure was identified from the following results.

¹H-NMR (CD₃COCD₃): δ [ppm]=1.34 to 1.65 (8H), 3.42 to 4.35 (46H)
¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Comparative Example 6

A compound represented by Formula (S) below was synthesized by the following method.

The same operation as in Example 3 was carried out except that the compound represented by HOCH₂CF₂OCF₂CF₂O)ₚCF₂CH₂OH (p indicating the average degree of polymerization in the formula is 2.5) (number average molecular weight: 468, molecular weight distribution: 1.1) was used instead of the compound represented by HOCH₂CF₂O(CF₂CF₂Oₘ(CF₂O)ₙCF₂CH₂OH (m and n indicating the average degree of polymerization in the formula are 2.5) in the first reaction and 4.47 g of a compound represented by Formula (25) below was used instead of the compound represented by Formula (14) in the second reaction, thereby obtaining 3.9 g (molecular weight: 2143, 1.8 mmol) of the compound represented by Formula (S) below.

The compound represented by Formula (25) was synthesized by introducing a THP group as a protective group into the primary and secondary hydroxyl groups of 3-allyloxy-1,2-propanediol and oxidizing the double bond of the obtained compound.

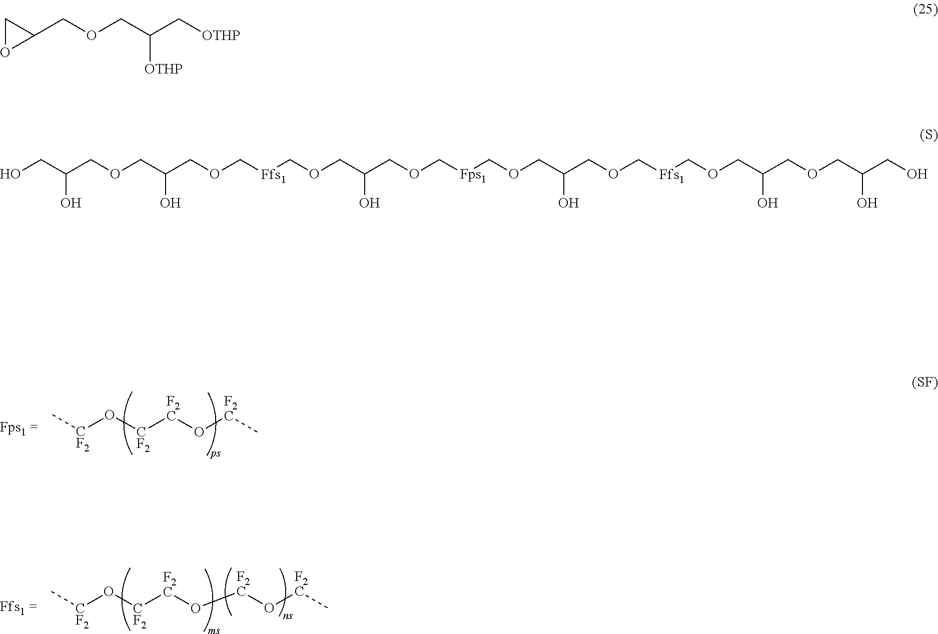

(In Formula (25), THP represents a tetrahydropyranyl group.)

(Fps₁ and Ffs₁ in Formula (S) are represented by Formula (SF), ps indicating the average degree of polymerization in Fps₁ is 2.5 and ms and ns indicating average degrees of polymerization in Ffs₁ each independently represent 2.5.)

¹H-NMR and ¹⁹F-NMR measurements of the obtained compound (S) were carried out, and the structure are was identified from the following results.

¹H-NMR (CD₃COCD₃): δ [ppm]=3:42 to 4.35 (50H)

¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Comparative Example 7

A compound represented by Formula (T) below was synthesized by the method described in Patent Document 2.

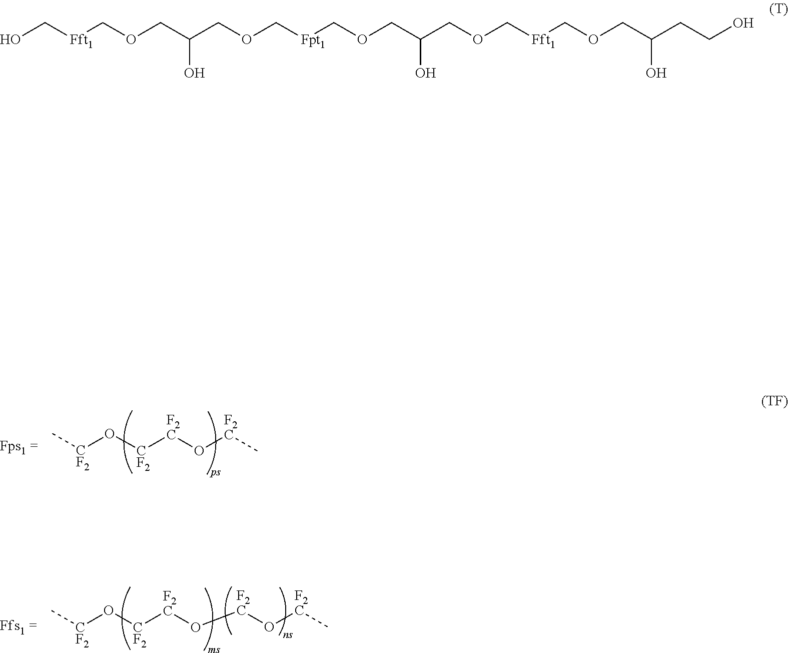

(Fpt₁ and Fft₁ in Formula (T) are represented by Formula (TF), pt indicating the average degree of polymerization in Fpt₁ 2.5, and out and mt indicating average degrees of polymerization in Fft₁ each independently represent 2.5.)

¹H-NMR and ¹⁹F-NMR measurements of the obtained compound (T) were carried out, and the structure was identified from the following results.

¹H-NMR (CD₃COCD₃): δ [ppm]=1.54 to 1.76 (2H), 3.42 to 4.28 (32H)

¹⁹F-NMR (CD₃COCD₃): δ [ppm]=−55.6 to −50.6 (10F), −77.7 (4F), −78.6 (2F), −80.3 (4F), −81.3 (2F), −90.0 to −88.5 (30F)

### Comparative Example 8

A compound represented by Formula (U) below was synthesized by the method described in Patent Document 1.

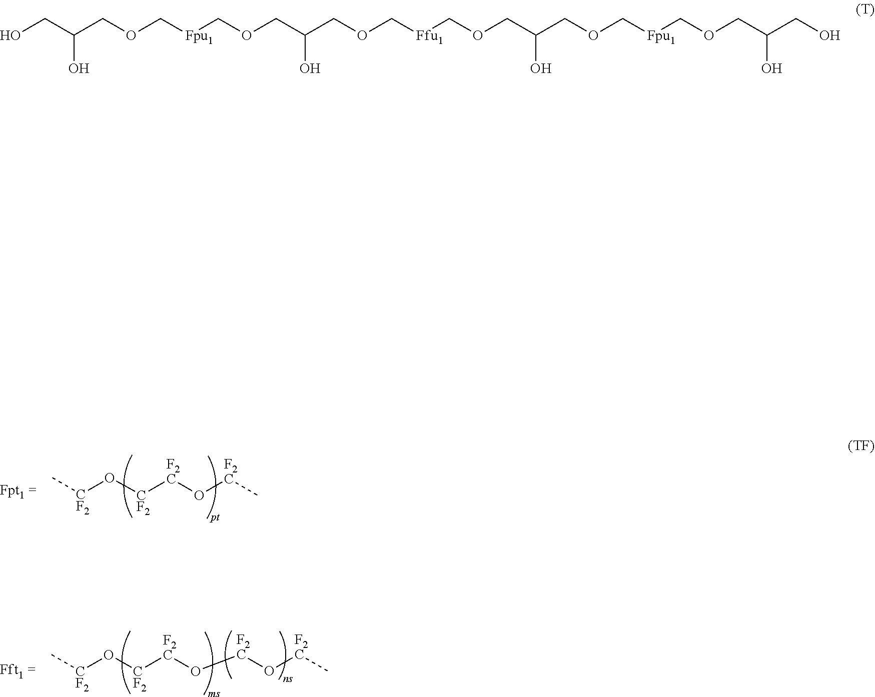

(Fpu₁ and Ffu₁ in Formula (U) are represented by Formula (UF), pa indicating the average degree of polymerization in Fpu₁ is 2.5, and mu and nu indicating average degrees of polymerization in Ffu₁ each independently represent 2.5.)

¹H-NMR and ¹⁹F-NMR measurements of the obtained compound (U) were carried out, and the structure was identified from the following results.

¹H-NMR (CD₃COCD₃): δ [ppm]=3.42 to 4.28 (38H)

¹⁹F-NMR (CD₃COCD₃): δ [ppm]=−55.6 to −50.6 (5F), −77.7 (2F), −78.6 (4F), −80.3 (2F), −81.3 (4F), −90.0 to −88.5 (30F)

### Comparative Example 9

A compound represented by Formula (V) below was synthesized by the method described in Patent Document 1.

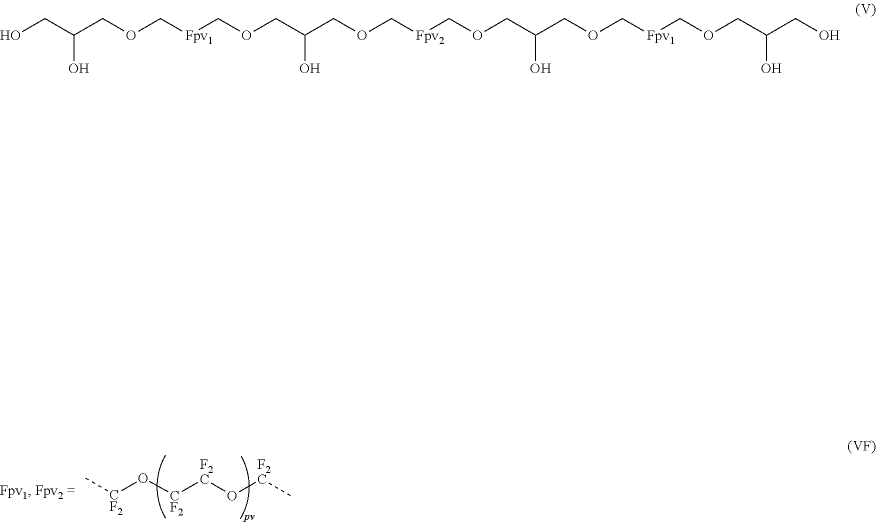

(Fpv₁ and Fpv₂ in Formula (V) are represented by Formula (VF), pv indicating the average degree of polymerization in Fpv₁ is 2.5, and pv indicating the average degree of polymerization in Fpv₂ represents 2.5.)

¹H-NMR and ⁹F-NMR measurements of the obtained compound (V) were carried out, and the structure was identified from the following results.

¹H-NMR (CD₃COCD₃): δ [ppm]=3.42 to 4.28 (38H)

¹⁹F-NMR (CD₃COCD₃): δ [ppm]=−78.6 (6F), −81.3 (6F), −90.0 to −88.5 (30F)

### Comparative Example 10

A compound represented by Formula (W) below was synthesized by the method described in Patent Document 1.

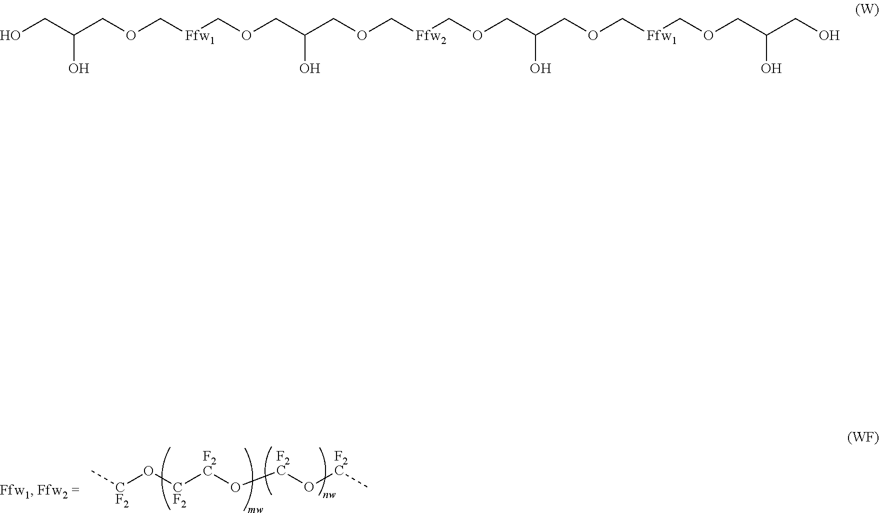

(Ffw₁ and Ffw₂ in Formula (W) are represented by Formula (WF), mw and nw indicating average degrees of polymerization in Ffw₁ are 2.5, and mw and nw indicating average degrees of polymerization in Ffw₂ represent 2.5.)

¹H-NMR and ¹⁹F-NMR measurements of the obtained compound (W) were carried out, and the structure was identified from the following results.

¹H-NMR (CD₃COCD₃): δ [ppm]=3.42 to 4.28 (38H)

¹⁹F-NMR (CD₃COCD₃): δ [ppm]=−55.6 to −50.6 (15F), −77.7 (6F), −80.3 (6F), −91.0 to −88.5 (30F)

### Comparative Example 11

A compound represented by Formula (X) below was synthesized by the method described in Patent Document 2.

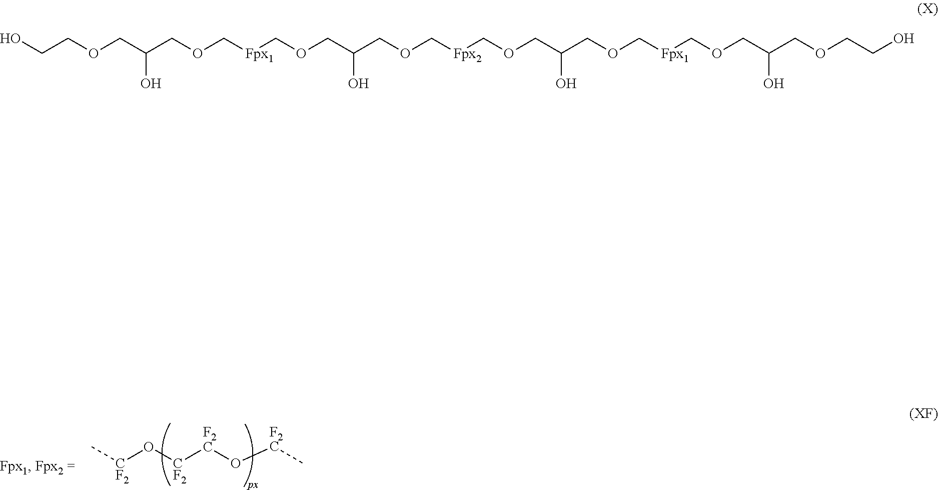

(Fpx₁ and Fpx₂ in Formula (X) are represented by Formula (XF), px indicating the average degree of polymerization in Fpx₁ 2.5, and px indicating the average degree of polymerization in Fpx₂ represents 2.5.)

¹H-NMR and ¹⁹F-NMR measurements of the obtained compound (X) were carried out, and the structure was identified from the following results.

¹H-NMR (CD₃COCD₃): δ [ppm]=¹H-NMR (CD₃COCD₃): δ [ppm]=3.46 to 4.18 (46H)

¹⁹F-NMR (CD₃COCD₃): δ [ppm]=−78.6 (6F), −81.3 (6F), −90.0 to −88.5 (30F)

### Comparative Example 12

A compound represented by Formula (Y) below was synthesized by the method described in Patent Document 2.

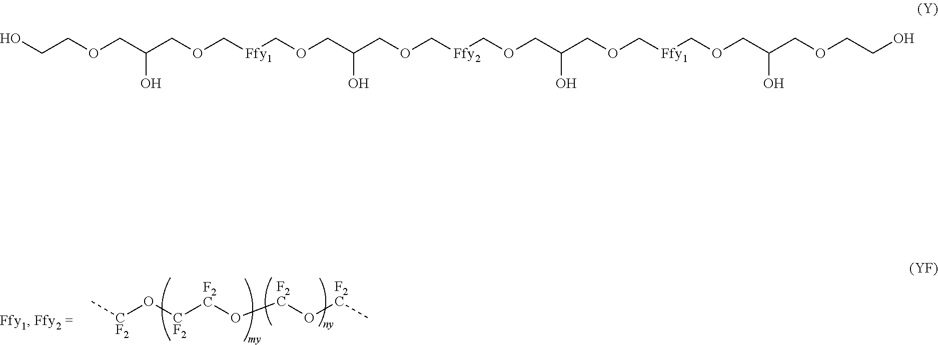

(Ffy₁ and Ffy₂ in Formula (Y) are represented by Formula (YF), may and ay indicating average degrees of polymerization in Ffy₁ are 2.5, and my and ny indicating average degrees of polymerization in Ffy₂ represent 2.5.)

¹H-NMR and ¹⁹F-NMR measurements of the obtained compound (Y) were carried out, and the structure was identified from the following results.

¹H-NMR (CD₃COCD₃): δ [ppm]=¹H-NMR (CD₃COCD₃): δ [ppm]=3.46 to 4.18 (46H)

¹⁹F-NMR (CD₃COCD₃): δ [ppm]=−55.6 to −50.6 (15F), −77.7 (6F), −80.3 (6F), −91.0 to −88.5 (30F)

### Comparative Example 13

A compound represented by Formula (Z) below was synthesized by the method described in Patent Document 2.

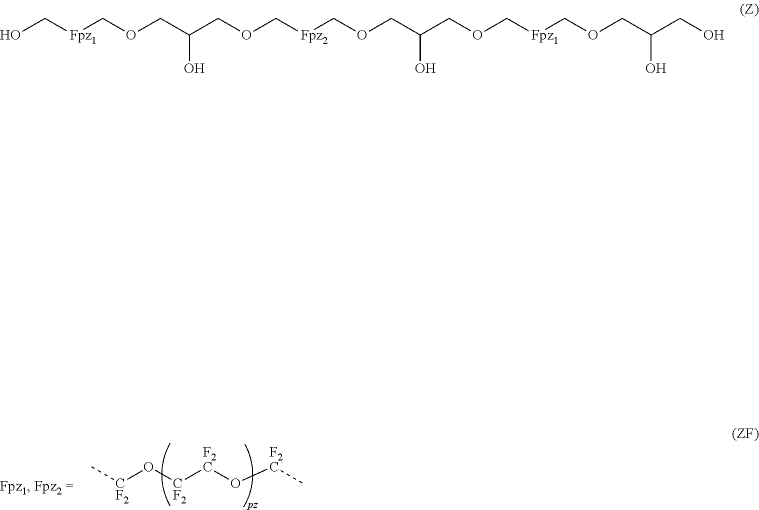

(Fpz₁ and Fpz₂ in Formula (7) are represented by Formula (ZF), pz indicating the average degree of polymerization in Fpz₁ is 2.5, and pz indicating the average degree of polymerization in Fpz₂ represents 2.5.)

¹H-NMR and ¹⁹F-NMR measurements of the obtained compound (Z) were carried out, and the structure was identified from the following results.

¹H-NMR (CD₃COCD₃): δ [ppm]=3.46 to 4.18 (32H)

¹⁹F-NMR (CD₃COCD₃): δ [ppm]=−78.6 (6F), −81.3 (6F), −90.0 to −88.5 (30F)

### Comparative Example 14

The compound represented by Formula (AA) below was synthesized by the following method.

14.0 g of the compound represented by HOCH₂CF₂O(CF₂CF₂O)ₚCF₂CH₂OH (p indicating the average degree of polymerization in the formula is 2.5) (number average molecular weight: 468, molecular weight distribution: 1.1), 2.34 g (molecular weight: 130.19, 18 mmol) of tert-butyl glycidyl ether, and 28 mL oft-butanol were added to a 200 mL eggplant flask in a nitrogen gas atmosphere and stirred until the mixture became uniform at room temperature. 1.0 g (molecular weight: 112.2, 9 mmol) of potassium tert-butoxide was further added to this uniform solution and reacted by being stirred at 70° C. for 16 hours.

A reaction product obtained after the reaction was cooled to 25° C., moved to #separatory funnel containing 100 ml of water, and extracted three times with 100 mL of ethyl acetate. An organic layer thereof was washed with water and dehydrated with anhydrous sodium sulfate. After the drying agent was filtered, the filtrate was concentrated, and the residue was purified by silica gel column chromatography, thereby obtaining 7.2 g of a compound represented by Formula (26) below (molecular weight: 598, 12.0 mmol).

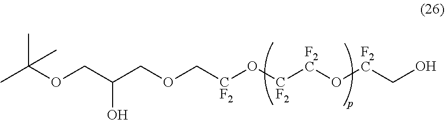

(In Formula (26), p indicating the average degree of polymerization is 2.5.)

7.2 g of the compound represented by Formula (26) (p indicating the average degree of polymerization in the formula is 2.5), 0.67 g of potassium tert-butoxide, and 10.5 mL of t-butanol were added to a 200 mL eggplant flask in a nitrogen gas atmosphere and stirred until the mixture became uniform at room temperature. 2.60 g of a compound represented by Formula (27) below was further added to this uniform liquid and reacted by being stirred at 70° ° C. for 16 hours.

The compound represented by Formula (27) was synthesized by oxidizing diallyl ether.

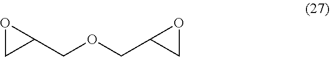

A reaction product obtained after the reaction was cooled to 25° ° C., moved to a separatory funnel containing 100 mL of water, and extracted three times with 100 ml of ethyl acetate. An organic layer thereof was washed with water and dehydrated with anhydrous sodium sulfate. After the drying agent was filtered, the filtrate was concentrated, and the residue was purified by silica gel column chromatography, thereby obtaining 5.9 g of a compound represented by Formula (28) below (molecular weight: 728, 7.7 mmol).

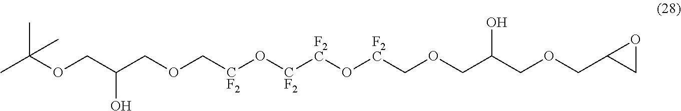 [SMILES: CC(C)(C)OCC(O)COCC(F)(F)OC(F)(F)C(F)(F)OC(F)(F)COCC(O)COCC1CO1]

(In Formula (28), p indicating the average degree of polymerization is 2.5.)

5.9 g of the compound represented by Formula (28) (p indicating the average degree of polymerization in the formula is 2.5), 0.12 g of potassium tert-butoxide, and 2.8 mL of t-butanol were added to a 200 mL eggplant flask in a nitrogen gas atmosphere and stirred until the mixture became uniform at room temperature. 1.6 g of the compound represented by HOCH₂CF₂O(CF₂CF₂O)ₚCF₂CH₂OH (p indicating the average degree of polymerization in the formula is 2.5) (number average molecular weight: 468, molecular weight distribution: 1.1) was further added to this uniform liquid and reacted by being stirred at 70° ° C. for 16 hours.

A reaction product obtained after the reaction was cooled to 25° ° C., moved to a separatory funnel containing 100 mL of water, and extracted three times with 100 mL of ethyl acetate. An organic layer thereof was washed with water and dehydrated with anhydrous sodium sulfate. After the drying agent was filtered, the filtrate was concentrated, and the residue was purified by silica gel column chromatography, thereby obtaining 3.3 g of a compound represented by Formula (AA) below (molecular weight: 1812, 2.4 mmol).

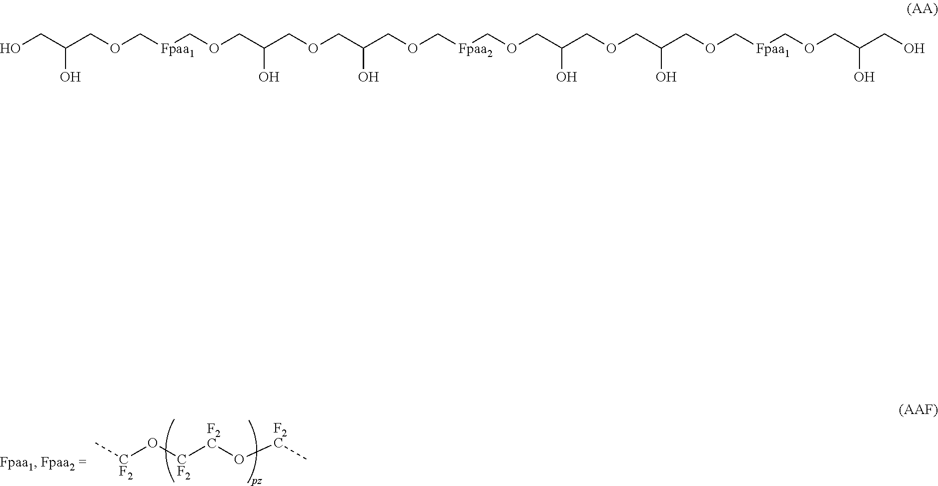

(Fpaa₁ and Fpaa₂ in Formula (AA) are represented by Formula (AAF), paa indicating the average degree of polymerization in Fpaa₁ is 2.5, and pas indicating the average degree of polymerization in Fpaa₂ represents 2.5.)

¹H-NMR and ¹⁹F-NMR measurements of the obtained compound (AA) were carried out, and the structure was identified from the following results.

¹H-NMR (CD₃COCD₃): δ [ppm]=3.47 to 4.24 (50H) ¹⁹F-NMR (CD₃COCD₃): δ [ppm]=−78.6 (6F), −81.3 (6F), −90.0 to −88.5 (30F)

Structures of R¹ and R⁷ (a in Formula (2-1), b in Formula (2-2), c in Formula (2-3), d in Formula (3-1), e in Formula (3-2), fin Formula (3-3) and Formula (3-4)), structures of R³ and R⁵ (g in Formula (4)), and structures of R², R⁴, and R⁶ (h and i in Formula (5), j in Formula (6), and k in Formula (7)) when the thus obtained compounds of Examples 1 to 13 and Comparative Examples 1 to 14 are applied to Formula (1) are shown in Tables 1 and 2.

TABLE 1

| | —R¹ | R², R [symbol] | R² | R [symbol] , R [symbol] | —R [symbol] | Compound |
|---|---|---|---|---|---|---|
| Example 1 | Formula (2-1) | Formula (6) | Formula (6) | Formula (4) | Same as —R [symbol] | (A) |
| | a = 2 | j = 2.5 | j = 2.5 | g = 1 | Same as —R [symbol] | |
| Example 2 | Formula (2-1) | Formula (7) | Formula (7) | Formula (4) | | (B) |
| | a = 3 | k = 2.5 | k = 2.5 | g = 1 | Same as —R [symbol] | |
| Example 3 | Formula (2-2) | Formula (5) | Formula (5) | Formula (4) | | (C) |
| | b = 2 | h = 2.5 | h = 2.5 | g = 1 | Same as —R [symbol] | |
| | | i = 2.5 | i = 2.5 | | | |
| Example 4 | Formula (2-2) | Formula (6) | Formula (6) | Formula (4) | | (D) |
| | b = 3 | j = 2.5 | j = 2.5 | g = 1 | Same as —R [symbol] | |
| Example 5 | Formula (3-2) | Formula (7) | Formula (7) | Formula (4) | | (E) |
| | c = 1 | k = 2.5 | k = 2.5 | g = 1 | Same as —R [symbol] | |
| Example 6 | Formula (3-2) | Formula (7) | Formula (7) | Formula (4) | | (F) |
| | c = 2 | k = 2.5 | k = 2.5 | g = 1 | Same as —R [symbol] | |
| Example 7 | Formula (2-3) | Formula (5) | Formula (5) | Formula (4) | | (G) |
| | c = 1 | h = 2.5 | h = 2.5 | g = 1 | Same as —R [symbol] | |
| | | i = 2.5 | i = 2.5 | | | |
| Example 8 | Formula (3-3) | Formula (5) | Formula (5) | Formula (4) | Same as —R [symbol] | (H) |
| | [symbol] = 1 | h = 2.5 | h = 2.5 | g = 1 | | |
| | | i = 2.5 | i = 2.5 | | | |
| Example 9 | Formula (3-1) | Formula (6) | Formula (6) | Formula (4) | Same as —R [symbol] | (I) |
| | d = 1 | j = 2.5 | j = 2.5 | g = 1 | | |
| Example 10 | Formula (3-1) | Formula (6) | Formula (6) | Formula (4) | Same as —R [symbol] | (J) |
| | d = 2 | j = 2.5 | j = 2.5 | g = 1 | | |
| Example 11 | Formula (3-3) | Formula (7) | Formula (7) | Formula (4) | Same as —R [symbol] | (K) |
| | [symbol] = 2 | k = 2.5 | k = 2.5 | g = 1 | | |
| Example 12 | Formula (3-4) | Formula (7) | Formula (7) | Formula (4) | Same as —R [symbol] | (L) |
| | | k = 2.5 | k = 2.5 | g = 1 | | |
| Example 13 | Formula (2-1) | Formula (7) | Formula (7) | Formula (4) | Same as —R [symbol] | (M) |
| | a = 1 | k = 2.5 | k = 2.5 | g = 1 | | |

[symbol]

indicates data missing or illegible when filed

TABLE 2

| | [symbol] R¹ | R², R [symbol] | R [symbol] | R [symbol] , R [symbol] | —R⁷ | Compound |
|---|---|---|---|---|---|---|
| Comparative Example 1 | 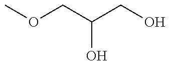 | Formula (5) h = 2.5 i = 2.5 | Formula (6) j = 2.5 | Formula (4) g = 1 | same as —R¹ | (N) |
| Comparative | Formula (2-1) | Formula (5) | Formula (6) | Formula (4) | same as —R¹ | (O) |
| Example 2 | a = 1 | h = 2.5 | j = 2.5 | g = 1 | | |
| | | i = 2.5 | | | | |
| Comparative Example 3 | 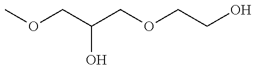 | Formula (5) h = 2.5 i = 2.5 | Formula (6) j = 2.5 | Formula (4) g = 1 | same as —R¹ | (P) |
| Comparative | Formula (2-2) | Formula (5) | Formula (6) | Formula (4) | same as —R¹ | (Q) |
| Example 4 | b = 2 | h = 2.5 | j = 2.5 | g = 1 | | |
| | | i = 2.5 | | | | |
| Comparative | Formula (2-2) | Formula (5) | Formula (6) | Formula (4) | same as —R¹ | (R) |
| Example 5 | b = 3 | h = 2.5 | j = 2.5 | g = 1 | | |
| | | i = 2.5 | | | | |
| Comparative Example 6 | 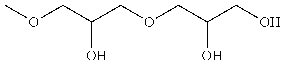 | Formula (5) h = 2.5 i = 2.5 | Formula (6) j = 2.5 | Formula (4) g = 1 | same as —R¹ | (S) |
| Comparative | —OH | Formula (5) | Formula (6) | Formula (4) | Formula (2-1) | (T) |
| Example 7 | | h = 2.5 | j = 2.5 | g = 1 | a = 1 | |
| | | i = 2.5 | | | | |
| Comparative Example 8 | 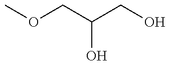 | Formula (6) j = 2.5 | Formula (5) h = 2.5 i = 2.5 | Formula (4) g = 1 | same as —R¹ | (U) |
| Comparative Example 9 | 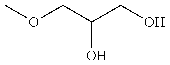 | Formula (6) j = 2.5 | Formula (6) j = 2.5 | Formula (4) g = 1 | same as —R¹ | (V) |
| Comparative Example 10 | 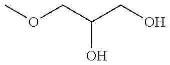 | Formula (5) h = 2.5 i = 2.5 | Formula (5) h = 2.5 i = 2.5 | Formula (4) g = 1 | same as —R¹ | (W) |
| Comparative Example 11 | 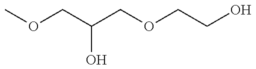 | Formula (6) j = 2.5 | Formula (6) j = 2.5 | Formula (4) g = 1 | same as —R¹ | (X) |
| Comparative Example 12 | 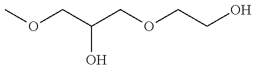 | Formula (5) h = 2.5 i = 2.5 | Formula (5) h = 2.5 i = 2.5 | Formula (4) g = 1 | same as —R¹ | (Y) |
| Comparative Example 13 | —OH | Formula (6) j = 2.5 | Formula (6) j = 2.5 | Formula (4) g = 1 | 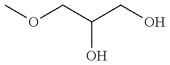 | (Z) |
| Comparative Example 14 | 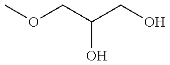 | Formula (6) j = 2.5 | Formula (6) j = 2.5 | Formula (4) g = 2 | same as —R¹ | (AA) |

[symbol]

indicates data missing or illegible when filed

In addition, the number average molecular weights (Mn) of the compounds of Examples 1 to 13 and Comparative Examples 1 to 14 were obtained by the above-described ¹H-NMR and ¹⁹F-NMR measurement. The results are shown in Table 3. It is inferred that, in the values of the average molecular weight of the synthesized compounds variations of approximately 1 to 5 may exist depending on, for example, the molecular weight distributions of the fluoropolyether used as a raw material of the compounds and differences in the operation at the time of synthesizing the compounds.

TABLE 3

| | | Number | | | | | |
|---|---|---|---|---|---|---|---|
| | | average | | | | | |
| | | molecular | Film | | | [symbol] | |
| | | weight | thickness | Corrosion resistance test | | resistance | Comprehensive |
| | Compound | (M [symbol] ) | (Å) | Burnished | Unburnished | test | evaluation |
| Example 1 | (A) | 1720 | 10 | A | A | B | B |
| Example 2 | (B) | 2424 | 10 | A | A | A | A |
| Example 3 | (C) | 2276 | 10 | B | B | B | B |
| Example 4 | (D) | 1809 | 10 | B | A | A | B |
| Example 5 | (E) | 2576 | 10 | A | A | A | A |
| Example 6 | (F) | 2604 | 11 | A | A | A | A |
| Example 7 | (G) | 2276 | 10 | A | A | A | A |
| Example 8 | (H) | 2424 | 10 | A | A | A | A |
| Example 9 | (I) | 1841 | 11 | A | A | A | A |
| Example 10 | (J) | 18 [symbol] | 10 | A | A | A | A |
| Example 11 | (K) | 2632 | 11 | A | A | A | A |
| Example 12 | (L) | 2544 | 10 | A | A | A | A |
| Example 13 | (M) | 2368 | 10 | B | B | B | B |
| Comparative | (N) | 1994 | 10 | [symbol] | [symbol] | D | [symbol] |
| Example 1 | | | | | | | |
| Comparative | (O) | 2023 | 10 | C | C | D | D |
| Example 2 | | | | | | | |
| Comparative | (P) | [symbol] | 10 | [symbol] | [symbol] | [symbol] | [symbol] |
| Example 3 | | | | | | | |
| Comparative | (Q) | 2111 | 10 | D | C | D | D |
| Example 4 | | | | | | | |
| Comparative | (R) | 213 [symbol] | 10 | C | [symbol] | C | C |
| Example 5 | | | | | | | |
| Comparative | (S) | 2143 | 11 | [symbol] | D | C | D |
| Example 6 | | | | | | | |
| Comparative | (T) | 1 [symbol] 34 | 10 | [symbol] | [symbol] | [symbol] | [symbol] |
| Example 7 | | | | | | | |
| Comparative | (U) | 1829 | 11 | [symbol] | [symbol] | D | [symbol] |
| Example 8 | | | | | | | |
| Comparative | (V) | 1 [symbol] 4 | 10 | [symbol] | [symbol] | C | D |
| Example 9 | | | | | | | |
| Comparative | (W) | 21 [symbol] | 10 | [symbol] | [symbol] | C | D |
| Example 10 | | | | | | | |
| Comparative | (X) | 1752 | 10 | D | D | D | D |
| Example 11 | | | | | | | |
| Comparative | (Y) | 2248 | 11 | D | D | D | D |
| Example 12 | | | | | | | |
| Comparative | (Z) | 1590 | 10 | [symbol] | [symbol] | [symbol] | [symbol] |
| Example 13 | | | | | | | |
| Comparative | (AA) | 1812 | 11 | [symbol] | [symbol] | C | D |
| Example 14 | | | | | | | |

[symbol]

indicates data missing or illegible when filed

Next, solutions for forming a lubricating layer were prepared using the compounds obtained in Examples 1 to 13 and Comparative Examples 1 to 14 by a method shown below. Moreover, lubricating layers of magnetic recording media were formed using the obtained solutions for forming a lubricating layer by a method shown below, and magnetic recording media of Examples 1 to 13 and Comparative Examples 1 to 14 were obtained.

### “Solutions for Forming Lubricating Layer”

The compounds obtained in Examples 1 to 13 and Comparative Examples 1 to 14 were each dissolved in VERTREL (registered trademark) XF (trade name, manufactured by Dupont-Mitsui Fluorochemicals Co., Ltd.), which is a fluorine-based solvent, diluted with VERTREL XF such that the film thicknesses of the coating films became 9 Å to 11 Å when applied onto protective layers, and used as solutions for forming a lubricating layer.

### “Magnetic Recording Media”

Magnetic recording media each having an adhesive layer, a soft magnetic layer, a first underlayer, a second underlayer, a magnetic layer, and a protective layer sequentially provided on a substrate having a diameter of 65 mm were prepared. As the protective layer, a carbon layer with a thickness of 1 to 5 nm was used.

The solutions for forming a lubricating layer of Examples 1 to 13 and Comparative Examples 1 to 14 were each applied by the dipping method onto the protective layers of the magnetic recording media in which the individual layers up to the protective layer had been formed. The dipping method was carried out under conditions of an immersion speed of 10 mm/sec, an immersion time of 30 seconds and a lifting speed of 1.2 mm/sec.

Thereafter, a burnishing step was performed in which burnishing tape holding abrasive grains having a grain size #6000 was scanned on the surface of each of the magnetic recording media on which the lubricating layer was formed.

The magnetic recording media after the burnishing step were placed in a thermostatic chamber at 120° ° C., to perform a heat treatment for 10 minutes.

Magnetic recording media (which were burnished) of Examples 1 to 13 and Comparative Examples 1 to 14 were obtained through the above-described steps.

In addition, magnetic recording media (which were unburnished) of Examples 1 to 13 and Comparative Examples 1 to 14 were obtained in the same manner as the burnished magnetic recording media except that a burnishing step was not performed.

### (Film Thickness Measurement)

The film thicknesses of the lubricating layers in the magnetic recording media (which were burnished and unburnished) of Examples 1 to 13 and Comparative Examples 1 to 14 thus obtained were measured using a Fourier transform infrared spectrophotometer (FT-IR) (trade name: Nicolet iS50, manufactured by Thermo Fisher Scientific Inc.). In all of the magnetic recording media of Examples 1 to 13 and Comparative Examples 1 to 14, there was no difference in the film thickness of the lubricating layer between being burnished and being unburnished. The results are shown in Table 3.

Next, corrosion resistance tests shown below were performed on the burnished and unburnished magnetic recording media of Examples 1 to 13 and Comparative Examples 1 to 14.

### (Corrosion Resistance Tests)

The magnetic recording media were exposed to conditions of a temperature of 85° C., and a relative humidity of 90% for 48 hours. Thereafter, munber of corroded spots of the magnetic recording media was counted using an optical surface analyzer and evaluated based on the following evaluation criteria. The results are shown in Table 3.

### “Evaluation Criteria”

A: Less than 150
B: Greater than or equal to 150 and less than 250
C: Greater than or equal to 250 and less than 350
D: Greater than or equal to 350 and less than 1.000
E: Greater than or equal to 1,000

Next, the burnished magnetic recording media of Examples 1 to 13 and

Comparative Examples 1 to 14 were subjected to a wear resistance test shown below.

### (Wear Resistance Test)

Using a pin-on-disk type friction and wear tester, an alumina ball with a diameter of 2 mm as a contactor was slid on a lubricating layer of each of the magnetic recording media at a load of 40 gf and a sliding speed of 0.25 m/sec, and the friction coefficient of the surface of the lubricating layer was measured. Then, the sliding time until the friction coefficient of the surface of the lubricating layer increased rapidly was measured. The sliding time until the friction coefficient increased rapidly was measured 4 times for the lubricating layer of each of the magnetic recording media, and the average value (time) thereof was used as an index of the wear resistance of the lubricant coating film. The evaluation criteria for friction coefficient increasing time were as follows. The evaluation results of the magnetic recording media in which the compounds of Examples 1 to 13 and Comparative Examples 1 to 14 are used are shown in Table 3.

### “Evaluation Criteria”

A: 650 sec or longer
B: 550 sec or longer and shorter than 650 sec
C: 450 sec or longer and shorter than 550 sec
D: 350 sec or longer and shorter than 450 sec
E: shorter than 350 sec

The time until the friction coefficient increased rapidly can be used as an index of the wear resistance of the lubricating layers due to reasons shown below. This is because the lubricating layers of the magnetic recording media wear as the magnetic recording media are used, and when the lubricating layers are lost due to wear, the contactors and the protective layers come into direct contact with each other, resulting in a sharp increase in the friction coefficient. It is thought that the time until this friction coefficient increases rapidly has a correlation with the friction test.

In addition, the magnetic recording media of Examples 1 to 13 and Comparative Examples 1 to 14 were comprehensively evaluated based on the criteria shown below. The results are shown in Table 3.

### (Comprehensive Evaluation)

A: All of the corrosion resistance test result for the burnished case, the corrosion resistance test result for the anburnished case, and the wear resistance test result are A.

B: All of the corrosion resistance test result for the burnished case, the corrosion resistance test result for the unburnished case, and the wear resistance test result are A or B, and any one or more thereof are B.

C: One of the corrosion resistance test result for the burnished case and the corrosion resistance test result for the unburnished case is B and the other is C, and the wear resistance test result is C.

D: All of the corrosion resistance test result for the burnished case, the corrosion resistance test result for the unburnished case, and the wear resistance test result are C or D, and any one or more thereof are D. Alternatively, the corrosion resistance test result for the burnished case and the corrosion resistance test result for the unburnished case are D or E, and the wear resistance test result is C.

E: The corrosion resistance test result for the burnished case and the corrosion resistance test result for the unburnished case are E, and the wear resistance test result is D or E.

As shown in Table 3, the comprehensive evaluations for the magnetic recording media of Examples 1 to 13 having a lubricating layer in which the compound represented by Formula (1) was used were A or B, which showed a favorable wear resistance and corrosion resistance. That is, the corrosion resistance test results for the magnetic recording media of Examples 1 to 13 in both cases where the magnetic recording media were subjected to and not subjected to tape burnishing were A or B, which showed favorable corrosion resistance. In addition, the magnetic recording media of Examples 1 to 13 had a long sliding time until the friction coefficient increased rapidly and the wear resistance test results were A or B, which showed a favorable wear resistance.

On the other hand, the comprehensive evaluation of Comparative Example 5 was C, the comprehensive evaluations of Comparative Examples 2, 4, 6, 9 to 12, and 14 were D, and the comprehensive evaluations of Comparative Examples 1, 3, 7, 8, and 13 were E. The comprehensive evaluations of the magnetic recording media of Comparative Examples 1 to 14 having a lubricating layer that did not contain the compound represented by Formula (1) were inferior to the magnetic recording media of Examples 1 to 13.

More specifically, it is inferred that, in all of the magnetic recording media of Examples 1, 2, 9, 10, 12, and 13 having a lubricating layer containing a compound in which a linking group between a carbon atom to which a terminal hydroxyl group is bound and a carbon atom to which a hydroxyl group adjacent to the terminal hydroxyl group is bound does not contain an ether bond in each of R¹ and R⁷ in Formula (1) and Examples 3 to 8 and 11 having a lubricating layer containing a compound in which the above described linking group contains an ether bond, favorable hydrophobicity is exhibited and water is prevented from intruding into the lubricating layer from outside, whereby a favorable corrosion suppressing effect was obtained. In addition, the lubricating layers in the magnetic recording media of Examples 1 to 13 contain a compound in which the distance between a terminal hydroxyl group and a hydroxyl group adjacent to the terminal hydroxyl group is appropriate. For this reason, it is inferred that intramolecular interaction between the hydroxyl groups is small and intramolecular aggregation is less likely to occur, whereby excellent adhesion properties with respect to the protective layer was obtained and an excellent wear resistance was exhibited.

For example. Examples 1 and 2 in which the number of atoms between a terminal hydroxyl group and a hydroxyl group adjacent to the terminal hydroxyl group in each of R¹ and R⁷ in Formula (1) is appropriate exhibited a favorable wear resistance compared to Comparative Example 9. Similarly, Example 3 in which the number of atoms between a terminal hydroxyl group and a hydroxyl group adjacent to the terminal hydroxyl group is appropriate exhibited a favorable wear resistance compared to Comparative Example 12. It is estimated that this may be because the number of atoms between the above-described hydroxyl groups in Examples 1 to 3 is appropriate, and therefore the mobility of compound molecules contained in the lubricating layers is appropriate, and the intramolecular aggregation between the above-described hydroxyl groups may be less likely to occur.

Furthermore, Examples 5, 6, and 8 to 12 in which R¹ and R⁷ in Formula (1) each independently contain three hydroxyl groups exhibited a particularly excellent corrosion suppressing effect and wear resistance. It is thought that this is because the three hydroxyl groups each contained in R¹ and R⁷ in Formula (1) exhibited excellent adhesion properties with respect to protective layers. If the adhesion properties between a lubricating layer and a protective layer are excellent, water can be prevented from intruding into the lubricating layer from outside.

On the other hand, in Comparative Example 6, although R¹ and R⁷ in Formula (1) each have three hydroxyl groups, the corrosion resistance test results were E (burnished) and D (unburnished), and the wear resistance test result was C. It is inferred that this may be because, in the compound contained in the lubricating layer in the magnetic recording medium of Comparative Example 6, the carbon atom to which a terminal hydroxyl group is bound is bound to a carbon atom to which a hydroxyl group adjacent to the terminal hydroxyl group is bound in R¹ and R⁷ in Formula (1). This is because, in the lubricating layer containing such a compound, either the terminal hydroxyl group or the hydroxyl group adjacent to the terminal hydroxyl group is oriented in the opposite direction with respect to a protective layer, and therefore adhesion of the hydroxyl groups with respect to the protective layer is less likely to be obtained.

In addition, in Example 13, the corrosion resistance test results were B (burnished) and B (unburnished), and the wear resistance test result was B. On the other hand, in Comparative Example 2 in which R¹ and R⁷ are the same as those in Example 13, the corrosion resistance test results were C (burnished) and C (unburnished), and the wear resistance test result was D.

In addition, in Example 3, the corrosion resistance test results were B (burnished) and B (unburnished), and the wear resistance test result was B. On the other hand, in Comparative Example 4 in which R¹ and R⁷ are the same as those in Example 3, the corrosion resistance test results were D (burnished) and C (unburnished), and the wear resistance test result was D.

In addition, in Example 4, the corrosion resistance test results were B (burnished) and A (unburnished), and the wear resistance test result was A. On the other hand, in Comparative Example 5 in which R¹ and R⁷ are the same as those in Example 4, the corrosion resistance test results were C (burnished) and B (unburnished), and the wear resistance test result was C.

In Comparative Examples 11 and 12, the corrosion resistance test results were D (burnished) and D (unburnished), and the wear resistance test result was D. On the other hand, in Comparative Example 3 in which R¹ and R⁷ are the same as those in Comparative Examples 11 and 12, the corrosion resistance test results were E (burnished) and E (unburnished), and the wear resistance test result was E.

From these results, it was found that a favorable corrosion suppressing effect and wear resistance were exhibited in a case where R², R⁴, and R⁶ in Formula (1) are the same as each other compared to a case where some of R², R⁴, and R⁶ are different from each other. It is thought that this is due to the following reasons.

In a compound in which R², R⁴, and R⁶ in Formula (1) are perfluoropolyether (PFPE) chains having the same structure, each PFPE chain in the molecule has the same mobility. On the other hand, in a compound in which some or all of R², R⁴, and R⁶ are PEPE chains having different structures, the difference in mobility of each PEPE chain in the molecule causes strain in the molecule. In a lubricating layer containing the compound in which R², R⁴, and R⁶ in Formula (1) are PEPE chains having the same structure, the compound molecule is not distorted, and the lubricating layer uniformly adheres closely to a protective layer. For this reason, it is inferred that the lubricating layer would have a high coating rate and favorable adhesion properties with respect to the protective layer, thereby obtaining a high corrosion suppressing effect and wear resistance.

### INDUSTRIAL APPLICABILITY

By using the lubricant for a magnetic recording medium containing the fluorine-containing ether compound of the present invention, a lubricating layer which has a high wear resistance and is highly effective in suppressing corrosion of a magnetic recording medium can be formed.

### REFERENCE SIGNS LIST

10 Magnetic recording medium
11 Substrate
12 Adhesive layer
13 Soft magnetic layer
14 First underlayer
Second underlayer
16 Magnetic layer
17 Protective layer
18 Lubricating layer

1. A fluorine-containing ether compound represented by Formula (1) below,
R¹—CH₂—R²—CH₂—R³—CH₂—R⁴—CH₂—R⁵—CH₂—R⁶—CH₂—R⁷ (1)
(in Formula (1), R², R⁴, and R⁶ are perfluoropolyether chains having the same structure, R³ and R⁵ are each independently a linking group containing one or more hydroxyl groups, and R¹ and R⁷ are each independently a terminal group represented by Formula (2) below), and
—O—CH₂—CH(OH)—([D]—CH(OH))ₛ-[E]-CH₂OH (2)
(in Formula (2), s is 0 or 1, [D] and [E] each independently is a chain structure consisting of a combination of 2 to 5 methylene groups (—CH₂—) and 1 oxygen atom (—O—) or a chain structure consisting of 1 to 4 methylene groups (—CH₂—), provided that, in a case where s is 0 and [E] contains an oxygen atom, the number of methylene groups contained in [E] is 3 or more).

2. The fluorine-containing ether compound according to claim 1,
wherein the terminal group represented by Formula (2) above is a terminal group represented by any of Formulae (2-1) to (2-3) and (3-1) to (3-4) below, 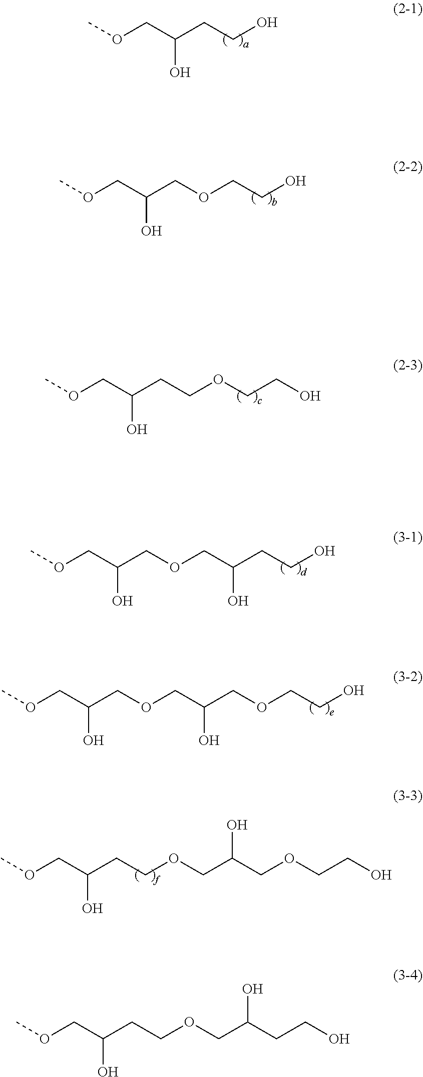
(a in Formula (2-1) is an integer of 1 to 4),
(b in Formula (2-2) is an integer of 2 to 3),
(c in Formula (2-3) is an integer of 1 to 3),
(d in Formula (3-1) is an integer of 1 to 4),
(e in Formula (3-2) is an integer of 1 to 3), and
(f in Formula (3-3) is an integer of 1 to 2).

3. The fluorine-containing ether compound according to claim 2,
wherein at least one of R¹ and R⁷ in Formula (1) above is the terminal group represented by any of Formula (3-1) to (3-4) above.

4. The fluorine-containing ether compound according to claim 1,
wherein R³ and R⁵ in Formula (1) above are linking groups represented by Formula (4) below, 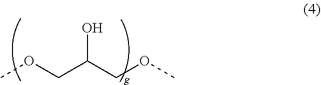
(g in Formula (4) is an integer of 1 to 4).

5. The fluorine-containing ether compound according to claim 1,
wherein R², R⁴, and R⁶ in Formula (1) above are any of Formulae (5) to (9) below,
—CF₂O—(CF₂CF₂O)ₕ—(CF₂O)ᵢ—CF₂— (5)
(in Formula (5), h and i indicate an average degree of polymerization and each independently represent 0.1 to 20),
—CF₂O—(CF₂CF₂O)ⱼ—CF₂— (6)
(in Formula (6), j indicates an average degree of polymerization and represents 0.1 to 20),
—CF₂CF₂O—(CF₂CF₂CF₂O)ₖ—CF₂CF₂— (7)
(in Formula (7), k indicates an average degree of polymerization and represents 0.1 to 20),
—CF₂CF₂CF₂O—(CF₂CF₂CF₂CF₂O)ₗ—CF₂CF₂CF₂— (8)
(in Formula (8), 1 indicates an average degree of polymerization and represents 0.1 to 10), and
—CF(CF₃)O—(CF₂CF(CF₃)O)ᵣ—CF(CF₃)— (9)
(in Formula (9), r indicates an average degree of polymerization and represents 0.1 to 20).

6. The fluorine-containing ether compound according to claim 1,
wherein R¹ and R⁷ in Formula (1) above are the same as each other.

7. The fluorine-containing ether compound according to claim 1,
wherein R³ and R⁵ in Formula (1) above are the same as each other.

8. The fluorine-containing ether compound according to claim 1,
wherein a number average molecular weight thereof is within a range of 500 to 10,000.

9. A lubricant for a magnetic recording medium comprising:
the fluorine-containing ether compound according to claim 1.

10. A magnetic recording medium,
wherein at least a magnetic layer, a protective layer, and a lubricating layer are sequentially provided on a substrate, and
wherein the lubricating layer contains the fluorine-containing ether compound according to claim 1.

11. The magnetic recording medium according to claim 10,
wherein an average film thickness of the lubricating layer is 0.5 nm to 2.0 nm.
